(19) United States
(12) Patent Application Publication
(10) Pub. No.: US 2025/0127870 A1
(43) Pub. Date: Apr. 24, 2025

(54) mRNA Vaccines Against Hantavirus

(71) Applicant: Board of Regents, The University of Texas System, Austin, TX (US)

(72) Inventors: Alexander Bukreyev, Galveston, TX (US); Mariano Garcia-Blanco, Galveston, TX (US); Ivan Kuzmin, Galveston, TX (US); Ruben Soto Acosta, Galveston, TX (US)

(21) Appl. No.: 18/691,787

(22) PCT Filed: Sep. 15, 2022
(86) PCT No.: PCT/US2022/043631

§ 371 Date: Mar. 13, 2024

Related U.S. Application Data
(60) Provisional application No. 63/245,101, filed on Sep. 16, 2021.

(51) Int. Cl. A61K 39/12; A61K 39/00; A61P 31/14; C12N 7/00

(52) U.S. Cl. CPC A61K 39/12; A61P 31/14; C12N 7/00; A61K 2039/53; A61K 2039/55555; A61K 2039/575; C12N 2760/12134

(57) ABSTRACT

One solution to the problem of Hantavirus pathology is design, production, and administration of a nucleic acid vaccine (NAV). In certain aspect the NAV is an mRNA vaccine. Certain embodiments are directed to the use of a polyprotein, which is cleaved to produce Gn (N-terminal) and Gc (C-terminal) glycoproteins, the Gn glycoprotein, the Gc glycoprotein, or the Gn and Gc glycoproteins hantaviruses as protective antigen(s) for development of hantavirus vaccines. The Gn/Gc protein, which is cleaved post-translationally to individual Gn and Gc proteins, can be used as an antigen for vaccines. In case of DNA and RNA-based vaccines, the complete M gene, which encodes the complete single open reading frame, which is cleaved post-translationally in the Gn and Gc proteins or individual open reading frames encoding either Gn or Gc, is used.

### PRIORITY PARAGRAPH

This application is an international application claiming priority to U.S. Provisional Patent Application 63/245,101 filed Sep. 16, 2021, which is incorporated herein by reference in its entirety.

### STATEMENT REGARDING FEDERALLY FUNDED RESEARCH

None.

### REFERENCE TO SEQUENCE LISTING

A sequence listing required by 37 CFR 1.821-1.825 is being submitted electronically with this application. The sequence listing is incorporated herein by reference. The sequence listing that is contained in the file named “UTMBP0404WO” which is 85.3 KB (as measured in Microsoft Windows®) and was created on Sep. 15, 2022.

### BACKGROUND

Hantaviruses cause human infections that can cause severe disease for which there are no effective vaccines or specific treatments. Hantaviruses are enveloped viruses with genomes that are composed of three segments of negative polarity RNA. The L (large) segment encodes the RNA-dependent RNA polymerase, which mediates transcription and replication of all three segments of the genome. The M (medium) RNA encodes a polyprotein which is cleaved to two envelope glycoproteins: Gn (N-terminal) and Gc (C-terminal). The S (small) RNA encodes the nucleocapsid (N) protein. A DNA vaccine which expresses the M gene of Puumala virus (an Old World hantavirus) induced virus-neutralizing antibody responses in vaccinated hamsters and non-human primates and protected hamsters against lethal infection with Puumala virus (PMID: 23239797). Similar data were generated with a DNA vaccine against Hantaan virus (an Old World hantavirus) (PMID:11507192). Experimental vaccines based on human replication-deficient adenovirus type V expressing either Gn, Gc, or N protected against Andes virus (a New World hantavirus) disease even though the virus neutralizing antibody responses were inconsistent (PMID: 19403663). In another study, a recombinant vesicular stomatitis virus (VSV)-based vaccine where the VSV G gene was replaced with the M gene of Andes virus was generated. The vaccine induced a neutralizing antibody response in hamsters and protected them from a lethal infection (PMID:21917979). In another study, VSV constructs in which the G gene was replaced with the M gene of Andes virus or Sin-Nombre virus (a New World hantavirus) induced neutralizing and cross-neutralizing antibody responses in vaccinated hamsters and conferred protection against death and disease caused by homologous and heterologous challenges (PMID: 31337019).

Polyclonal antibodies induced by vaccination with DNA vaccines encoding Gn/Gc proteins of Hantaan or Puumala virus were protective against infection of hamsters with the respective viruses (PMID: 32508764). Neutralizing monoclonal antibodies against the Gn and Gc proteins of the Andes virus protected Andes virus-infected hamsters against lethality when administered on days 3 and 8 post-infection in two studies (PMID: 32209676, 33951434).

There remains a need for improved and effective Hantavirus vaccines, particularly for humans.

### SUMMARY

One solution to the problem of Hantavirus infection is design, production, and administration of a nucleic acid vaccine (NAV). In certain aspect the NAV is an mRNA vaccine. Certain embodiments are directed to the use of (i) a polyprotein, which is cleaved to produce Gn (N-terminal) and Gc (C-terminal) glycoproteins, (ii) the Gn glycoprotein, (iii) the Gc glycoprotein, or (iv) the Gn and Gc glycoproteins of Old World and New World hantaviruses as protective antigen(s) for development of vaccines against Hantaviruses. The Gn/Gc protein (envelope polyprotein), which is cleaved post-translationally to individual Gn and Gc proteins, can be used as an antigen for vaccines based on any replication-competent and replication-deficient viral vectors. Alternatively, one open reading frame encoding either Gn or Gc can be used. In case of DNA and RNA-based vaccines, the complete M gene, which encodes the complete single open reading frame, which is cleaved post-translationally in the Gn and Gc proteins or individual open reading frames encoding either Gn or Gc, can be used.

Embodiments include, but are not limited to at least three platforms or constructs configured for expression of Gn (e.g., SEQ ID NO:4), Gc (e.g., SEQ ID NO:5), or Gn and Gc open reading frame (ORF)(e.g., SEQ ID NO:2).

In one aspect, a platform or construct is configured as a linear nucleic acid DNA or mRNA with one ORF encoding Gn and Gc (e.g., nucleotides 144 to 3496 of SEQ ID NO:1 DNA or 114 to 3466 SEQ ID NO:13 RNA) separated by a protease cleavage site (e.g., encoded by nucleotides 2021-2035 of SEQ ID NO:1 or 1991-2005 SEQ ID NO:13) that produces a protein that is cleaved in the cell expressing the ORF into a Gn polypeptide (encoded by nucleotides 144 to 2020 of SEQ ID NO:1 or 114 to 1990 of SEQ ID NO:13) and Gc polypeptide (encoded by nucleotides 2036 to 3496 of SEQ ID NO:1 or 2006 to 3466 of SEQ ID NO:13). In certain aspects the cleavage site has an amino acid sequence of WAASA (SEQ ID NO:10). UTRs, a can be modified to enhance expression in a target cell, e.g., dendritic cells (DCs). The cleavage can be carried out by a cellular peptide complex (signalase) or similar mechanism. In certain aspects, the linear moncistronic platform or construct includes a promoter appropriately positioned 5′ to the ORF. The promoter can be a T7 promoter for example, such as the one present at nucleotides 11 to 27 of SEQ ID NO:1. The linear monocistronic platform or construct can also include a 5′ UTR that can be positioned between the promoter and 5′ to the ORF. As a non-limiting example a UTR can have a sequence of nucleotides 31 to 82 of SEQ ID NO:1 or 1 to 52 of SEQ ID NO:13. In certain aspects the ORF can encode a polyprotein comprising Gn and Gc. The ORF can include a N-terminal signal peptide, for example an ANDV signal peptide which is encoded at the 5′ end of the ORF by nucleotides 83 to 143 of SEQ ID NO:1 or 53 to 113 of SEQ ID NO:13. The platform or construct can include a 3′ UTR. An example of a 3′ UTR is encoded by nucleotides 3500 to 3780 of SEQ ID NO:1 or 3470 to 3750 of SEQ ID NO:13. The 3′ terminus can include a poly adenylation segment. An example of such a poly adenylation segment is provided from nucleotides 3781 to 3942 of SEQ ID NO:1 or 3751 to 3912 of SEQ ID NO:13. In certain aspects, the construction comprises a nucleotide sequence having 80, 85, 90, 95, 98, 99, to 100% identity to the nucleic acid sequence of SEQ ID NO:1 or SEQ ID NO:13, starting from nucleotide 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 to any nucleotide between nucleotide 3781/3751 to nucleotide 3942/3912, respectively.

In another aspect, a platform or construct is configured as a linear nucleic acid, DNA (SEQ ID NO:3) or mRNA (SEQ ID NO:14), with two ORFs, a Gn ORF and a Gc ORF. The linear nucleic acid contains an internal ribosome entry site (IRES) between the Gn ORF and Gc ORF forming a bicistronic construct. In certain aspects, both ORFs can include an amino terminal signal peptide [SP](amino acids encoded by nucleotides 83 to 154 and 2780 to 2851 of SEQ ID NO:3 or 33 to 124 and 2750 to 2821 of SEQ ID NO: 14). The platform or construct can include a 5′cap-5′UTR-ORF 1-17 [SP][biscistronic reporter sequences including IRES]-ORF2 1-17[SP]651-end-3′UTR. In certain aspects, the linear bicistronic platform or construct includes a promoter appropriately positioned 5′ to the ORF. The promoter can be a T7 promoter for example, such as the one present at nucleotides 11 to 27 of SEQ ID NO:3. The linear bicistronic platform or construct can also include a 5′ UTR that can be positioned between the promoter and 5′ to the ORF. As a non-limiting example, a 5′ UTR can have a sequence of nucleotides 31 to 82 of SEQ ID NO:3 or 1 to 32 of SEQ ID NO:14. In certain aspects the first ORF (ORF1) can encode a Gn polypeptide and the second ORF (ORF2) can encode a Gc polypeptide. The N-terminal signal peptide can be an ANDV signal peptide or a signal peptide having a similar function. An IRES can be operatively position between ORF1 and ORF2, in certain aspects the IRES has a nucleotide sequence 2039 to 2779 of SEQ ID NO:3 or 2009 to 2749 of SEQ ID NO:14. In certain aspects the IRES is a CVB3 IRES or a sequence with a similar functionality. The platform or construct can include a 3′ UTR. An example of a 3′ UTR is encoded by nucleotides 4316 to 4593 of SEQ ID NO:3 or 4286 to 4563 of SEQ ID NO:14. The 3′ terminus can include a poly adenylation segment. An example of such a poly adenylation segment is provided from nucleotides 4594 to 4758 of SEQ ID NO:3 or 4564 to 4728 of SEQ ID NO:14. In certain aspects, the construct or nucleic acid comprises a nucleotide sequence having 80, 85, 90, 95, 98, 99, to 100% identity to the nucleic acid sequence of SEQ ID NO:3 or SEQ ID NO:14, starting from nucleotide 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 to any nucleotide between and including nucleotide 4594/4564 to nucleotide 4758/4728, respectively.

In a third aspect, a platform or construct is configured as a circular nucleic acid, crcDNA (SEQ ID NO:6) or mRNA (SEQ ID NO:15) having an IRES (see for example PMID: 30902547) or a variation of the circular nucleic acid with two IRES driving translation of Gn and Gc ORFs. The termini may be spliced using an autocatalytic reaction as described in PMID 29980667 or by ligation.

In certain aspects, a moncistronic circRNA platform or construct includes a promoter appropriately positioned 5′ to the ORF. The promoter can be a T7 promoter for example (e.g., nucleotides 3 to 19 of SEQ ID NO:6). The monocistronic circRNA includes a 5′ external homology segment (e.g., nucleotides 33 to 52 of SEQ ID NO:6 or 13 to 32 of SEQ ID NO:15) and 5′ internal homology segment (e.g., nucleotides 235 to 254 of SEQ ID NO:6 or 236 to 255 of SEQ ID NO:15) flanking a 3′ intron and exon fragment (e.g., nucleotides 53 to 234 of SEQ ID NO:6 or 33 to 214 of SEQ ID NO:15) that is position 5′ of a first polyAC spacer segment (e.g., nucleotides 255 to 304 of SEQ ID NO:6 or 235 to 284 of SEQ ID NO:15). The first polyAC spacer segment is positioned 5′ to an IRES segment (e.g., nucleotides 305 to 1045 of SEQ ID NO:6 or 285 to 1025 of SEQ ID NO:15). The IRES segment is 5′ to the G protein ORF (e.g., nucleotides 1046 to 4462 of SEQ ID NO:6 or 1026 to 4442 of SEQ ID NO:15). The G protein ORF encodes a polyprotein having a signal peptide encoded by nucleotides 1046 to 1117 or 1026 to 1097 and a protease cleavage site (e.g., encoded by nucleotides 2984 to 2998 of SEQ ID NO:6 or 2964 to 2978 of SEQ ID NO:15). The polyprotein when expressed in a cell is cleaved, as described above, into Gn polypeptide (e.g., encoded by nucleotides 1118 to 2983 of SEQ ID NO:6 or 1098 to 2963 of SEQ ID NO:15) and Gc polypeptide (e.g., encoded by nucleotides 2999 to 4462 of SEQ ID NO:6 or 2979 to 4442 of SEQ ID NO:15). In certain aspects, the N-terminal signal peptide can be, for example, an ANDV signal peptide a second polyAC region is positioned 3′ to the ORF (e.g., nucleotides 4463 to 4482 of SEQ ID NO:6 or 4443 to 4462 of SEQ ID NO:15). Positioned 3′ to the polyAC region is a 3′ internal homology segment (e.g., nucleotides 4483 to 4503 of SEQ ID NO:6 or 4463 to 4485 of SEQ ID NO:15) and a 3′ external homology segment (e.g., nucleotides 4634 to 4653 of SEQ ID NO:6 or 4614 to 4633 of SEQ ID NO:15) flanking a 5′ intron and exon fragment (e.g., nucleotides 4504 to 4633 of SEQ ID NO:6 or 4484 to 4613 of SEQ ID NO:15). In certain aspects, the construction or nucleic acid comprises a nucleotide sequence having 80, 85, 90, 95, 98, 99, to 100% identity to the nucleic acid sequence of SEQ ID NO:6 or SEQ ID NO:15, starting from nucleotide 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 to any nucleotide between and including nucleotide 4653/4633 to nucleotide 4687/4667, respectively.

In certain aspects, the bicistronic circRNA platform or construct includes a promoter appropriately positioned 5′ to the ORF. The promoter can be a T7 promoter for example (e.g., nucleotides 3 to 19 of SEQ ID NO:7). The bicistronic circRNA includes a 5′ external homology segment (e.g., nucleotides 33 to 52 of SEQ ID NO:7 or 14 to 33 of SEQ ID NO:16) and 5′ internal homology segment (e.g., nucleotides 235 to 254 of SEQ ID NO:7 or 216 to 235 of SEQ ID NO:16) flanking a 3′ intron and exon fragment (e.g., nucleotides 53 to 234 of SEQ ID NO:7 or 34 to 215 of SEQ ID NO:16) that is position 5′ of a first polyAC spacer segment (e.g., nucleotides 255 to 304 of SEQ ID NO:7 or 236 to 285 of SEQ ID NO:16). The first polyAC spacer segment is positioned 5′ to a first IRES segment (e.g., nucleotides 305 to 1045 of SEQ ID NO:7 or 286 to 1026 of SEQ ID NO:16). The first IRES segment is 5′ to the first ORF (ORF1) (e.g., nucleotides 1046 to 3001 of SEQ ID NO:7 or 1027 to 2982 of SEQ ID NO:16). The ORF1 encodes a Gn protein having a signal peptide encoded by nucleotides 1046 to 1117 of SEQ ID NO:7 or 1027 to 1098 of SEQ ID NO:16. A second IRES segment is 3′ to the first ORF (ORF1) (e.g., nucleotides 3002 to 3742 of SEQ ID NO:7 or 2983 to 3723 of SEQ ID NO:16). The second IRES is 5′ to second ORF (ORF2) (e.g., nucleotides 3743 to 5278 of SEQ ID NO:7 or 3724 to 5259 of SEQ ID NO:16). ORF2 encodes a Gc protein having a signal peptide encoded by nucleotides 3743 to 3814 of SEQ ID NO:7 or 3724 to 3795 of SEQ ID NO:16. A second polyAC segment (e.g., nucleotides 5279 to 5298 of SEQ ID NO:7 or 5260 to 5279 of SEQ ID NO:16) is 3′ to ORF2. A 3′ internal homology segment (e.g., nucleotides 5299 to 5319 of SEQ ID NO:7 or 5280 to 5300 of SEQ ID NO:16), which is 3′ to the second polyAC, and 3′ external homology segment (e.g., nucleotides 5450 to 5469 of SEQ ID NO:7 or 5431 to 5450 of SEQ ID NO:16) flanking a 5′ intron and exon fragment (e.g., nucleotides 5320 to 5449 of SEQ ID NO:7 or 5301 to 5430 of SEQ ID NO:16). In certain aspects, the N-terminal signal peptide can be, for example, an ANDV signal peptide In certain aspects, the construction comprises a nucleotide sequence having 80, 85, 90, 95, 98, 99, to 100% identity to the nucleic acid sequence of SEQ ID NO:7 or SEQ ID NO:16, starting from nucleotide 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 to any nucleotide between and including nucleotide 5469/5450 to nucleotide 5503/5484, respectively.

In certain aspects, a moncistronic Gn circRNA platform or construct includes a promoter appropriately positioned 5′ to the ORF. The promoter can be a T7 promoter for example (e.g., nucleotides 3 to 19 of SEQ ID NO:8, RNA transcript begin at about nucleotide 20). The Gn circRNA includes a 5′ external homology segment (e.g., nucleotides 33 to 52 of SEQ ID NO:8) and 5′ internal homology segment (e.g., nucleotides 235 to 254 of SEQ ID NO:8) flanking a 3′ intron and exon fragment (e.g., nucleotides 53 to 234 of SEQ ID NO:8) that is position 5′ of a first polyAC spacer segment (e.g., nucleotides 255 to 304 of SEQ ID NO:8). The first polyAC spacer segment is positioned 5′ to an IRES segment (e.g., nucleotides 305 to 1045 of SEQ ID NO:8). The IRES segment is 5′ to the Gn protein ORF (e.g., nucleotides 1046 to 2998 of SEQ ID NO:8). The Gn protein ORF encodes a protein having a signal peptide encoded by nucleotides 1046 to 1117. In certain aspects, the N-terminal signal peptide can be, for example, an ANDV signal peptide a second polyAC region is positioned 3′ to the ORF (e.g., nucleotides 3001 to 3021 of SEQ ID NO:8). Positioned 3′ to the polyAC region is a 3′ internal homology segment (e.g., nucleotides 3022 to 3042 of SEQ ID NO:8) and a 3′ external homology segment (e.g., nucleotides 3173 to 3192 of SEQ ID NO:8) flanking a 5′ intron and exon fragment (e.g., nucleotides 3043 to 3172 of SEQ ID NO:8). In certain aspects, the construction comprises a nucleotide sequence having 80, 85, 90, 95, 98, 99, to 100% identity to the nucleic acid sequence of SEQ ID NO:8, starting from nucleotide 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 to any nucleotide between and including nucleotide 3193 to nucleotide 3226.

In certain aspects, a moncistronic Gc circRNA platform or construct includes a promoter appropriately positioned 5′ to the ORF. The promoter can be a T7 promoter for example (e.g., nucleotides 3 to 19 of SEQ ID NO:9). The Gc circRNA includes a 5′ external homology segment (e.g., nucleotides 33 to 52 of SEQ ID NO:9, with the RNA transcript beginning at about nucleotide 20) and 5′ internal homology segment (e.g., nucleotides 235 to 254 of SEQ ID NO:9) flanking a 3′ intron and exon fragment (e.g., nucleotides 53 to 234 of SEQ ID NO:9) that is position 5′ of a first polyAC spacer segment (e.g., nucleotides 255 to 304 of SEQ ID NO:9). The first polyAC spacer segment is positioned 5′ to an IRES segment (e.g., nucleotides 305 to 1045 of SEQ ID NO:9). The IRES segment is 5′ to the Gc protein ORF (e.g., nucleotides 1046 to 2578 of SEQ ID NO:9). The Gc protein ORF encodes a protein having a signal peptide encoded by nucleotides 1046 to 1117. In certain aspects, the N-terminal signal peptide can be, for example, an ANDV signal peptide. A second polyAC region is positioned 3′ to the ORF (e.g., nucleotides 2582 to 2601 of SEQ ID NO:9). Positioned 3′ to the polyAC region is a 3′ internal homology segment (e.g., nucleotides 2602 to 2622 of SEQ ID NO:9) and a 3′ external homology segment (e.g., nucleotides 2753 to 2772 of SEQ ID NO:9) flanking (positioned on either side) a 5′ intron and exon fragment (e.g., nucleotides 2623 to 2752 of SEQ ID NO:9). In certain aspects, the construction comprises a nucleotide sequence having 80, 85, 90, 95, 98, 99, to 100% identity to the nucleic acid sequence of SEQ ID NO:9, starting from nucleotide 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 to any nucleotide between and including nucleotide 2773 to nucleotide 2806.

The platforms/constructs/NAVs can include various modifications to improve protective efficacy of the vaccines including but not limited to one or more of:

(i) Sequences that enhance translation and mRNA stability in dendritic cells (DCs) to enhance antigen presentation (e.g., 3 ′UTR sequences) can be included in the platform/construct. For example, as described in PMID: 16940422, a 120-nucleotide-long poly(A), an unmasked poly(A) tail with a free 3′ end rather than one extended with unrelated nucleotides, and/or 2 sequential β-globin 3′ untranslated regions. Since it is also possible that mRNA vaccines are translated in myocytes and antigens subsequently transferred to DCs (PMID: 33477534) mRNA sequences from highly stable and translated human myocyte mRNAs (e.g., myosin) can be included.

(ii) Optimization of codons for enhanced mammalian translation, which can include substitution of rare codons with codons more frequently used by mammalian (human and NHP) species.

(iii) Substitution of adenosine with the N6-methyladenosine (m6A) cap (PMID:33536170) to evade the host innate immunity and improve the translation.

(iv) Methylation of RNA to evade induction of interferon response (see for example PMID: 33536170) and thereby increase translation.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 illustrates schematics of RNA vaccine platforms. Elements of the Platform A example construct include: (i) T7 promoter; (ii) 5′ UTR from Andes virus; (iii) ORF (codon-optimized using GenScript online tool) with the following features: Enrichment of GC content (56%); Use of frequent codons for expression in human cells; Avoidance of restriction sites for Not-I, Eco-RV and BstBI enzymes used in construct preparation; (iv) 3′ UTR from concatenated sequences of human mtRNR I and AES 3′ UTRs (PMID:30638957). (v) Poly-A tail (120 adenosines), followed with Eco-RV restriction site, and BstBI restriction site after additional 30A. Platform C, D, and E illustrate circular mRNA platforms. Circular mRNA with IRES or a variation of the circular mRNA with two IRES driving translation of Gn and Gc ORFs. The termini may be spliced using an autocatalytic reaction as described in PMID 29980667 or by ligation. Platform E illustrated circular mRNA encoding Gn and Gc individually and separately.

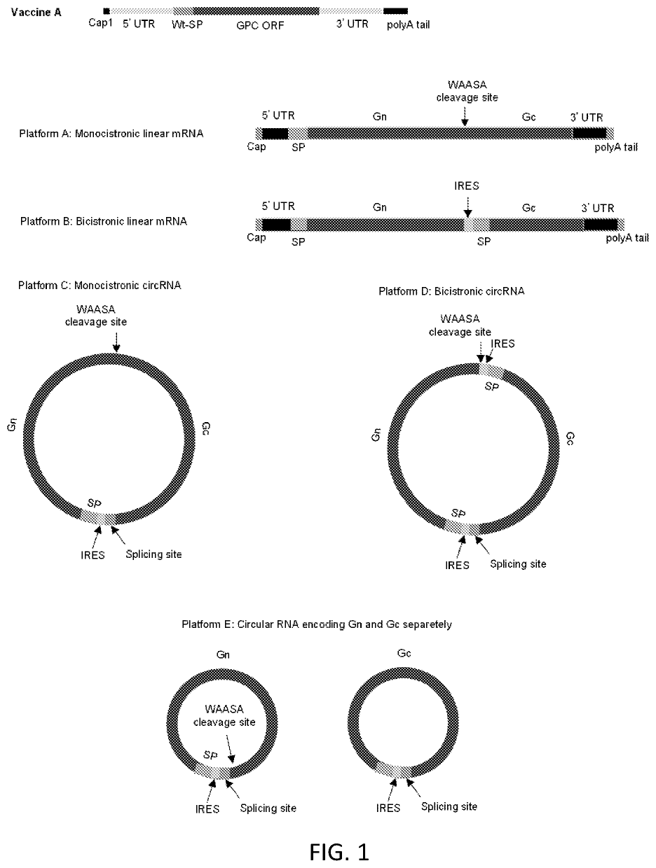

FIG. 2A-2F. ANDV Gn/Gc expression in transfected cells. A549 cells were transfected with mRNA constructs, and 24 h late subjected to flow cytometry with ANDV Gn/Gc-specific monoclonal antibodies and FICT-labeled secondary antibody or to qRT-PCR for cytokine expression. (A) Histograms of flow-cytometry data for cells transfected with U-mRNA and m1Ψ-mRNA; left bars show proportions of GFP-negative cells, right bars show proportions of GFP-positive cells. (B) Mean proportions of GFP-positive cells for the same mRNA constructs. (C) Mean fluorescence intensity (MFI) determined for the same cells. (D) Fold changes in IFNβ gene expression normalized on GAPDH. (E) electron microscopy of virus-like particles in supernatant of 293T cells transfected with ANDV mRNA. Immunostaining with primary human ANDV antibody cocktail and secondary 6 nm colloidal gold anti-human antibody, followed with fixation and uranyl acetate counterstain. ** p<0.01. (F) Represnetative schematic of one example of a DNA template. Restriction sites NotI and BstBI were used for insertion of the construct into pUC-19 vector whereas EcoRV and BstBI sites (separated by a 30 bp poly-A spacer) were used for linearization of the template for in-vitro transcription. Triplet GGC after T7 promotor inserted for improved transcription efficiency. 5′ terminus resembles the original ANDV sequence. Open reading frame was codon-optimized (GenScript) and followed with 3′ terminus designed from two concatenated human mitochondrial sequences, mtRNR1 and AES. The sequence was terminated with poly-A tail of 120 bp.

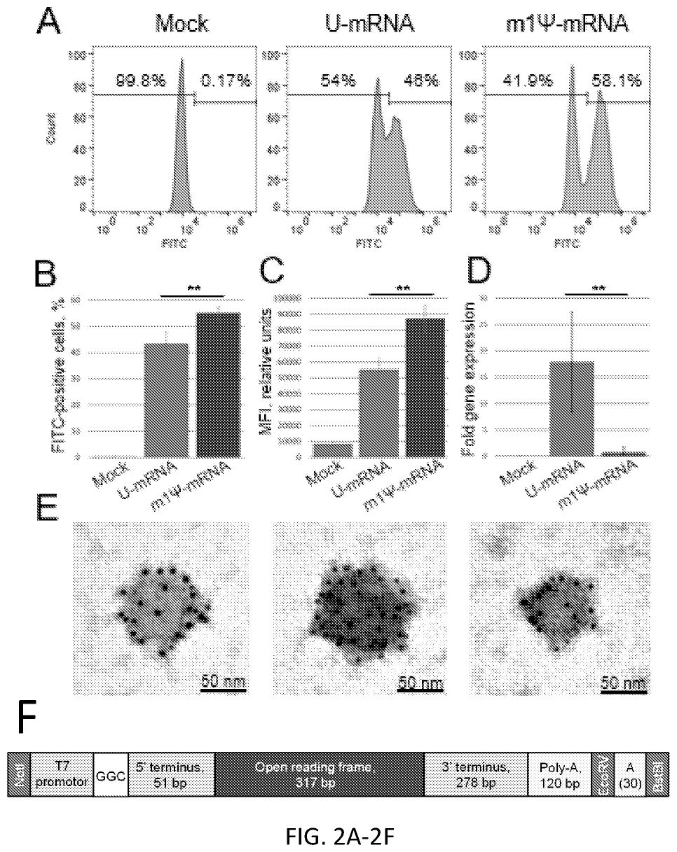

FIG. 3A-3E. ANDV mRNA vaccine elicits antibody response and protects Syrian hamsters from lethal challenge. (A) Schematic representation of the experiment. Animals were vaccinated twice on days 0 and 21, challenged on day 42, and the survivors were euthanized on day 28 post challenge (dpc). Serum was collected at indicated time points, and organs at necropsy. (B) ANDV Gn/Gc-IgG-ELISA; (C) ANDV-neutralizing antibodies; (D) SNV-neutralizing antibodies; (E) PUUV—N-IgG-ELISA. PRNT50 indicates 50% plaque reduction-neutralization titer. Terminal serum was collected from control (mock) animals on 9 dpc (because of clinical disease and euthanasia), and on 28 dpc from other animals. *, p<0.05; *** p<0.001.

FIG. 4A-4B. Syrian hamster survival study. (A) Schematic representation of the experiment. (B) Survival of Syrian hamsters vaccinated intramuscularly with regular (non-modified uridine) or modified (N1-pseudouridine) monocistronic linear Andes vaccine constructs, and challenged intramuscularly with Andes virus.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

A current interest in the fields of therapeutics and diagnostics is the ability and methods for designing, synthesizing, and delivering a nucleic acid to effect physiologic outcomes beneficial to a cell, a tissue, an organ and ultimately to a subject. The nucleic acid can be a ribonucleic acid (RNA) such as a messenger RNA (mRNA) encoding a peptide or polypeptide of interest. One beneficial outcome is the intracellular translation of the nucleic acid and production of at least one encoded peptide or polypeptide of interest.

Of particular interest, is the ability to design, synthesize and deliver a nucleic acid, such as a ribonucleic acid (RNA) which encodes an antigen for the purpose of vaccination.

Described herein are compositions (including pharmaceutical compositions) and methods for the design, preparation, manufacture, formulation, and/or use of nucleic acid vaccines (NAVs) where at least one component of the NAV is a nucleic acid molecule. In particular, described herein are compositions (including pharmaceutical compositions) and methods for the selection, design, preparation, manufacture, formulation, and/or use of nucleic acid vaccines (NAVs) where at least one component of the NAV is a polynucleotide, a RNA polynucleotide, and/or a mRNA which encodes an antigen derived from an infectious microorganism, in particular Hantavirus. Also provided are systems, processes, devices and kits for the selection, design and/or utilization of the NAVs described herein.

### I. NUCLEIC ACID VACCINES (NAVS)

Nucleic Acid Vaccines (NAVs) described herein comprise one or more polynucleotides (platform or construct) which encode one or more Hantavirus antigens. Polynucleotide constructs include antigen-encoding RNA polynucleotides such as mRNAs. The polynucleotide constructs can include at least one chemical modification. The sequences provided can be the sense strand of a sequence but one of skill would readily identify the complementary anti-sense sequence as well. Also, the nucleotide sequences may be presented as DNA sequences, deoxyribose adenine, guanine, thymine, cytosine (AGTC) and/or RNA sequences ribose adenine, guanine, uracil, cytosine (AGUC); one of skill would readily identify the RNA or DNA counterpart.

NAV compositions of the invention may comprise other components including, but not limited to, adjuvants. Adjuvants may also be administered with or in combination with one or more NAVs. In one aspect, an adjuvant acts as a co-signal to prime T-cells and/or B-cells and/or NK cells as to the existence of an infection. Adjuvants may be co-administered by any route, e.g., intramusculary, subcutaneous, IV or intradermal injections. Adjuvants useful in the present invention may include, but are not limited to, natural or synthetic adjuvants. Adjuvants can be selected from any of the classes (1) mineral salts, e.g., aluminium hydroxide and aluminium or calcium phosphate gels; (2) emulsions including: oil emulsions and surfactant based formulations, e.g., microfluidised detergent stabilized oil-in-water emulsion, purified saponin, oil-in-water emulsion, stabilized water-in-oil emulsion; (3) particulate adjuvants, e.g., virosomes (unilamellar liposomal vehicles incorporating influenza haemagglutinin), structured complex of saponins and lipids, polylactide co-glycolide (PLG); (4) microbial derivatives; (5) endogenous human immunomodulators; and/or (6) inert vehicles, such as gold particles; (7) microorganism derived adjuvants; (8) tensoactive compounds; (9) carbohydrates; or combinations thereof.

Specific adjuvants may include, without limitation, cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, AS04, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumer™, Algal Glucan, Bay R1005, Theramide®, Stearyl Tyrosine, Specol, Algammulin, Avridine®, Calcium Phosphate Gel, CTA 1-DD gene fusion protein, DOC/Alum Complex, Gamma Inulin, Gerbu Adjuvant, GM-CSF, GMDP, Recombinant hIFN-gamma/Interferon-g, Interleukin-1l, Interleukin-2, Interleukin-7, Sclavo peptide, Rehydragel LV, Rehydragel HPA, Loxoribine, MF59, MTP-PE Liposomes, Murametide. Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, ASO3, Quil-A vaccine adjuvant, RC529 vaccine adjuvant, LTR1920 Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant. AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-E112K, and/or Matrix-S. Other adjuvants which may be co-administered with the NAVs of the invention include, but are not limited to interferons, TNF-alpha, TNF-beta, chemokines such as CCL21, eotaxin, HMGB1, SA100-8alpha, GCSF, GMCSF, granulysin, lactoferrin, ovalbumin, CD-40L, CD28 agonists, PD-1, soluble PD1, L1 or L2, or interleukins such as IL-1, IL-2, IL-4, IL-6, IL-7, IL-10. IL-12, IL-13, IL-21. IL-23, IL-15, IL-17, and IL-18. These may be administered with the NAV on the same encoded polynucleotide, e.g., polycistronic, or as separate mRNA encoding the adjuvant and antigen.

NAVs of the present invention may vary in their valency. Valency refers to the number of antigenic components in the NAV polynucleotide. In some embodiments, the NAVs are monovalent (monocistronic). In some embodiments, the NAVs are divalent (bicistronic). The antigenic components of the NAVs may be on a single polynucleotide or on separate polynucleotides.

An “effective amount” of the NAV composition is provided based, at least in part, on the target tissue, target cell type, means of administration, physical characteristics of the polynucleotide (e.g., size, and extent of modified nucleosides) and other components of the NAV, and other determinants. In general, an effective amount of the NAV composition provides an induced or boosted immune response as a function of antigen production in the cell.

Activation of the Immune Response. According to various embodiments, the NAVs comprising the polynucleotides disclosed herein may act as a vaccine. As used herein, a “vaccine” refers to a composition, for example, a substance or preparation that stimulates, induces, causes or improves immunity in an organism, e.g., a mammalian organism (a human, etc.). Preferably, a vaccine provides immunity against one or more diseases or disorders, including prophylactic and/or therapeutic immunity. NAVs may be administered prophylactically or therapeutically as part of an active immunization scheme to healthy individuals or early in infection during the incubation phase or during active infection after onset of symptoms.

The use of RNA in or as a vaccine overcomes the disadvantages of conventional genetic vaccination involving incorporating DNA into cells in terms of safeness, feasibility, applicability, and effectiveness to generate immune responses. RNA molecules are considered to be significantly safer than DNA vaccines, as RNAs are more easily degraded. They are cleared quickly out of the organism and cannot integrate into the genome and influence the cell's gene expression in an uncontrollable manner. It is also less likely for RNA vaccines to cause severe side effects like the generation of autoimmune disease or anti-DNA antibodies (Bringmann A. et al., Journal of Biomedicine and Biotechnology (2010), vol. 2010). Transfection with RNA requires only insertion into the cell's cytoplasm, which is easier to achieve than into the nucleus. However, RNA is susceptible to RNase degradation and other natural decomposition in the cytoplasm of cells.

In one embodiment, the polynucleotides of the NAVs of the invention may be administrated with other prophylactic or therapeutic compounds. As a non-limiting example, the prophylactic or therapeutic compound may be an adjuvant or a booster. As used herein, when referring to a prophylactic composition, such as a vaccine, the term “booster” refers to an extra administration of the prophylactic composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In certain aspects, the polynucleotides of the NAVs of the invention may be administered intranasally, intramuscularly, or intradermally.

The administration of a NAV described herein may be in combination with an anti-viral agent. Examples of anti-viral agents include, but are not limited to, abacavir (ZIAGEN®), abacavir/lamivudine/zidovudine (Trizivir®), aciclovir or acyclovir (CYCLOVIR®, HERPEX®, ACIVIR®, ACIVIRAX®, ZOVIRAX®, ZOVIR®), adefovir (Preveon®, Hepsera®), amantadine (SYMMETREL®), amprenavir (AGENERASE®), ampligen, arbidol, atazanavir (REYATAZ®), boceprevir, cidofovir, darunavir (PREZISTA®), delavirdine (RESCRIPTOR®), didanosine (VIDEX®), docosanol (ABREVA®), edoxudine, efavirenz (SUSTIVA®, STOCRIN®), emtricitabine (EMTRIVA®), emtricitabine/tenofovir/efavirenz (ATRIPLA®), enfuvirtide (FUZEON®), entecavir (BARACLUDE®, ENNAVIR®), famciclovir (FAMVIR®), fomivirsen (VITRAVENE®), fosamprenavir (LEXIVA®, TELZIR®), foscarnet (FOSCAVIR®), fosfonet, ganciclovir (CYTOVENE®, CYMEVENE®, VITRASERT®), GS 9137 (ELVITEGRAVIR®), imiquimod (ALDARA®, ZYCLARA®, BESELNA®), indinavir (CRIXIVAN®), inosine, inosine pranobex (EVIUNOVIR®), interferon type 1, interferon type 11, interferon type III, kutapressin (NEXAVIR®), lamivudine (ZEFFIX®, HEPTOVIR®, EPIVIR®), lamivudinelzidovudine (COMBIVIR®), lopinavir, loviride, maraviroc (SELZENTRY®, CELSENTRI®), methisazone, MK-2048, moroxydine, nelfinavir (VIRACEPT®), nevirapine (VIRAMUNE®), oseltamivir (TAMIFLU®), peginterferon alfa-2a (PEGASYS®), penciclovir (DENAVIR®), peramivir, pleconaril, podophyllotoxin (CONDYLOX®), raltegravir (ISENTRESS®), ribavirin (COPEGUs, REBETOL®, RIBASPHERE®, VILONA® AND VIRAZOLE®), rimantadine (FLUMADINE®), ritonavir (NORVIR®), pyramidine, saquinavir (INVIRASE®, FORTOVASE®), stavudine, tea tree oil (melaleuca oil), tenofovir (VIREAD®), tenofovir/emtricitabine (TRUVADA®), tipranavir (APTIVUS®), trifluridine (VIROPTIC®), tromantadine (ViRU-MERZ®), valaciclovir (VALTREX®), valganciclovir (VALCYTE®), vicriviroc, vidarabine, viramidine, zalcitabine, zanamivir (RELENZA®), and zidovudine (azidothymidine (AZT). RETROVIR®, RETROVIS®).

In certain aspects, NAVs are can be used as memory booster vaccines and are administered to boost antigenic memory across a time period of 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more years.

### II. NAV POLYNUCLEOTIDES

According to certain embodiments, the polynucleotides encode at least one polypeptide of interest (an antigen or immunogen). Antigens of the present invention may be wild type derived from Hantavirus or modified, engineered, designed or artificial. They may have any combination of the features described herein. In certain embodiments, the antigen is derived from the M segment of a Hantavirus, in particular the antigen is all or a portion of the glycoprotein polypeptide, including Gn, Gc, or Gn and Gc or fragments thereof.

Certain embodiments are directed to nucleic acid molecules that encode one or more peptides or polypeptides of interest. Such peptides or polypeptides serve as an antigen or antigenic molecule. The term “nucleic acid,” in its broadest sense, includes any compound and/or substance that comprise a polymer of nucleotides. These polymers are often referred to as polynucleotides. Nucleic acids or polynucleotides of the invention include, but are not limited to, ribonucleic acids (RNAs), which may or may not include ribonucleotide analogs or modifications.

In certain aspects, the polynucleotides of the present invention that are circular are known as “circular polynucleotides” or “circP.” As used herein, “circular polynucleotides” or “circP” means a single stranded circular polynucleotide which acts substantially like, and has the properties of, an RNA. The term “circular” is also meant to encompass any secondary or tertiary configuration of the circP.

In some embodiments, the polynucleotide includes from about from 30 to 50, from 30 to 100, from 30 to 250, from 30 to 500, from 30 to 1,000, from 30 to 1,500, from 30 to 3,000, from 30 to 5,000, from 100 to 250, from 100 to 500, from 100 to 1,000, from 100 to 1,500, from 100 to 3,000, from 100 to 5,000, from 500 to 1,000, from 500 to 1,500, from 500 to 2,000, from 500 to 3,000, from 500 to 5,000, from 1,000 to 1,500, from 1,000 to 2,000, from 1.000 to 3,000, from 1,000 to 5,000, from 1,500 to 3,000, from 1,500 to 5,000, from 2,000 to 3,000, from 2,000 to 5,000.

In one embodiment, the polynucleotides of the present invention may encode at least one peptide or polypeptide of interest.

In one embodiment, the length of a region encoding at least one polypeptide of interest of the polynucleotides present invention is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000, 4,000, 5,000, 6,000, 7,000 nucleotides). As used herein, such a region may be referred to as a “coding region” or “region encoding” or “open reading frame (ORF)”.

In one embodiment, the polynucleotides of the present invention is or functions as a messenger RNA (mRNA). As used herein, the term “messenger RNA” (mRNA) refers to any polynucleotide which encodes at least one peptide or polypeptide of interest and which is capable of being translated to produce the encoded peptide polypeptide of interest in vitro, in vivo, in situ or ex vivo.

In one embodiment, the polynucleotides of the present invention may be structurally modified or chemically modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

In certain aspects, the polynucleotides have a uniform chemical modification of all or any of the same nucleoside type or a population of modifications, or a measured percent of a chemical modification of all any of the same nucleoside type but with random incorporation, such as where all uridines are replaced by a uridine analog, e.g., pseudouridine. In another embodiment, the polynucleotides may have a uniform chemical modification of two, three, or four of the same nucleoside type throughout the entire polynucleotide (such as all uridines and all cytosines, etc. are modified in the same way).

When the polynucleotides of the present invention are chemically and/or structurally modified the polynucleotides may be referred to as “modified polynucleotides.”

In certain aspects, the polynucleotides of the present invention may include a sequence encoding a self-cleaving peptide. The self-cleaving peptide may be, but is not limited to, a 2A peptide. As a non-limiting example, the 2A peptide may have the protein sequence: GSGATNFSLLKQAGDVEENPGP (SEQ ID NO:11), fragments or variants thereof. In one embodiment, the 2A peptide cleaves between the last glycine and last proline. As another non-limiting example, the polynucleotides of the present invention may include a polynucleotide sequence encoding the 2A peptide having the protein sequence GSGATNFSLLKQAGDVEENPGP (SEQ ID NO:11) fragments or variants thereof. One such polynucleotide sequence encoding the 2A peptide is GGAAGCGGAGCTACTAACTTCAGCCTGCTGAAGCAGGCTGGAGACGTGGAGGAGAA CCCTGGACCT (SEQ ID NO:12). The polynucleotide sequence of the 2A peptide may be modified or codon optimized by the methods described herein and/or are known in the art.

Polynucleotide Architecture. Traditionally, the basic components of an mRNA molecule include at least a coding region, a 5′ UTR, a 3′ UTR, a 5′ cap and a poly-A tail. The polynucleotides described herein may function as mRNA.

Circular Polynucleotide Architecture. Certain aspects are directed to polynucleotides which are circular or cyclic. As the name implies circular polynucleotides are circular in nature meaning that the termini are joined in some fashion, whether by ligation, covalent bond, common association with the same protein or other molecule or complex or by hybridization. The circular polynucleotides or circPs that encode at least one peptide or polypeptide of interest are known as circular RNAs or circRNA. The antigens of the NAVs of the present invention may be encoded by one or more circular RNAs or circRNAs.

As used herein, “circular RNA” or “circRNA” means a circular polynucleotide that can encode at least one peptide or polypeptide of interest.

In order to further enhance protein production, polynucleotides of the present invention can be designed to be conjugated to other polynucleotides, dyes, intercalating agents (e.g., acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG], polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug. In a preferred embodiment, the polynucleotides of the present invention which encode an antigen are conjugated to one or more dendritic cell markers. Conjugation may result in increased stability and/or half life and may be particularly useful in targeting the polynucleotides to specific sites in the cell, tissue or organism.

As used herein, “polypeptide” means a polymer of amino acid residues (natural or unnatural) linked together most often by peptide bonds. The term, as used herein, refers to proteins, polypeptides, and peptides of any size, structure, or function. In one embodiment, the polypeptides of interest are antigens encoded by the polynucleotides as described herein.

“Substitutional variants” when referring to polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to polypeptides are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to polypeptides are those with one or more amino acids in the native or starting amino acid sequence removed. Ordinarily, deletional variants will have one or more amino acids deleted in a particular region of the molecule.

“Covalent derivatives” when referring to polypeptides include modifications of a native or starting protein with an organic proteinaceous or non-proteinaceous derivatizing agent, and/or post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

As used herein the terms “termini” or “terminus” when referring to polypeptides refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

In some embodiments, the encoded polypeptide variant may have the same or a similar activity as the reference polypeptide (e.g., Gn, Gc, or Gn and Gc). Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.) Other tools are described herein, specifically in the definition of “Identity.”

Default parameters in the BLAST algorithm include, for example, an expect threshold of 10, Word size of 28, Match/Mismatch Scores 1, −2, Gap costs Linear. Any filter can be applied as well as a selection for species specific repeats, e.g., Homo sapiens.

Cell-Penetrating Polypeptides. The polynucleotides disclosed herein may also encode one or more cell-penetrating polypeptides. As used herein, “cell-penetrating polypeptide” or CPP refers to a polypeptide which may facilitate the cellular uptake of molecules. A cell-penetrating polypeptide of the present invention may contain one or more detectable labels. The polypeptides may be partially labeled or completely labeled throughout. The polynucleotides may encode the detectable label completely, partially or not at all. The cell-penetrating peptide may also include a signal sequence. As used herein, a “signal sequence” refers to a sequence of amino acid residues bound at the amino terminus of a nascent protein during protein translation. The signal sequence may be used to signal the secretion of the cell-penetrating polypeptide.

In one embodiment, the polynucleotides may also encode a fusion protein. The fusion protein may be created by operably linking a heterologous protein or peptide to a therapeutic protein. As used herein, “operably linked” refers to the therapeutic protein and the heterologous protein or peptide being connected in such a way to permit the expression of the complex when introduced into the cell. Preferably, the therapeutic protein may be covalently linked to the heterologous protein or peptide in the formation of the fusion protein.

Polynucleotides Having Untranslated Regions (UTRs). The polynucleotides of the present invention (e.g., antigen-encoding polynucleotides featured in the NAVs of the invention) may comprise one or more regions or parts which act or function as an untranslated region. Where polynucleotides are designed to encode at least one polypeptide of interest, the polynucleotides may comprise one or more of these untranslated regions.

By definition, untranslated regions (UTRs) of a gene are transcribed but not translated. In mRNA, the 5′ UTR starts at the transcription start site and continues to the start codon but does not include the start codon; whereas, the 3′ UTR starts immediately following the stop codon and continues until the transcriptional termination signal. The regulatory features of UTR can be incorporated into the polynucleotides of the present invention to among other things, enhance the stability of the molecule.

Natural 5′ UTRs bear features which play roles in translation initiation. They harbor signatures like Kozak sequences which are commonly known to be involved in the process by which the ribosome initiates translation of many genes. 5′ UTR also have been known to form secondary structures which are involved in elongation factor binding. By engineering the features typically found in abundantly expressed genes of specific target organs, one can enhance the stability and protein production of the polynucleotides of the invention.

Other non-UTR sequences may also be used as regions or subregions within the polynucleotides. For example, introns or portions of introns sequences may be incorporated into regions of the polynucleotides of the invention. Incorporation of intronic sequences may increase protein production as well as polynucleotide levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes.

A UTR from various gene(s) may be incorporated into the regions of the polynucleotide. Furthermore, multiple UTRs of any known gene may be utilized. It is also within the scope of the present invention to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In one embodiment, flanking regions are selected from a family of transcripts whose proteins share a common function, structure, feature of property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

3′ UTR and the AU Rich Elements. Natural or wild type 3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes: Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif c-Jun and Myogenin are two well-studied examples of this class.

Regions Having a 5′ Cap. The 5′ cap structure of a natural mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or ante-terminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

In some embodiments, polynucleotides may be designed to incorporate a cap moiety. Modifications to the polynucleotides of the present invention may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, Mass.) may be used with α-thio-guanosine nucleotides according to the manufacturer's instructions to create a phosphorothioate linkage in the 5′-ppp-5′ cap. Additional modified guanosine nucleotides may be used such as α-methyl-phosphonate and seleno-phosphate nucleotides.

Additional modifications include, but are not limited to, 2′-O-methylation of the ribose sugars of 5′-terminal and/or 5′-anteterminal nucleotides of the polynucleotide (as mentioned above) on the 2′-hydroxyl group of the sugar ring. Multiple distinct 5′-cap structures can be used to generate the 5′-cap of a nucleic acid molecule, such as a polynucleotide which functions as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs may be chemically (i.e. non-enzymatically) or enzymatically synthesized and/or linked to the polynucleotides of the invention.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′ mppp-G; which may equivalently be designated 3′ 0-Me-m7G(5′)ppp(5′)G). The 3′-0 atom of the other, unmodified, guanine becomes linked to the 5′-terminal nucleotide of the capped polynucleotide. The N7- and 3′-O-methylated guanine provides the terminal moiety of the capped polynucleotide.

Another example of a cap is mCAP, which is similar to ARCA but has a 2′-O-methyl group on guanosine (i.e., N7,2′-O-dimethyl-guanosine-5′-triphosphate-5′-guanosine, m7Gm-ppp-G).

Viral Sequences. Additional viral sequences such as, but not limited to, the translation enhancer sequence of the barley yellow dwarf virus (BYDV-PAV), the Jaagsiekte sheep retrovirus (JSRV) and/or the Enzootic nasal tumor virus (See e.g., International Pub. No. WO2012129648; herein incorporated by reference in its entirety) can be engineered and inserted in the polynucleotides of the invention and can stimulate the translation of the construct in vitro and in vivo. Transfection experiments can be conducted in relevant cell lines at and protein production can be assayed by ELISA at 12 hr, 24 hr, 48 hr, 72 hr and day 7 post-transfection.

IRES Sequences. Further, provided are polynucleotides (e.g., antigen-encoding polynucleotides featured in the NAVs of the invention) which may contain an internal ribosome entry site (IRES). First identified as a feature Picorna virus RNA, IRES plays an important role in initiating protein synthesis in absence of the 5′ cap structure. An IRES may act as the sole ribosome binding site, or may serve as one of multiple ribosome binding sites of an mRNA. Polynucleotides containing more than one functional ribosome binding site may encode several peptides or polypeptides that are translated independently by the ribosomes (“multicistronic nucleic acid molecules”). When polynucleotides are provided with an IRES, further optionally provided is a second translatable region. Examples of IRES sequences that can be used according to the invention include without limitation, those from coxsackievirus B3 (CVB3), picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

Poly-A Tails. During RNA processing, a long chain of adenine nucleotides (poly-A tail) may be added to a polynucleotide such as an mRNA molecule in order to increase stability. Immediately after transcription, the 3′ end of the transcript may be cleaved to free a 3′ hydroxyl. Then poly-A polymerase adds a chain of adenine nucleotides to the RNA. The process, called polyadenylation, adds a poly-A tail that can be between, for example, approximately 80 to approximately 250 residues long, including approximately 80, 90, 100, 110, 120, 130, 140, 150, 160, 170, 180, 190, 200, 210, 220, 230, 240 or 250 residues long.

According to the present invention, terminal groups on the poly A tail may be incorporated for stabilization into polynucleotides of the invention (e.g., antigen-encoding polynucleotides featured in the RNAVs of the invention). Polynucleotides of the present invention may include des-3′ hydroxyl tails. They may also include structural moieties or 2′-Omethyl modifications as taught by Junjie Li, et al. (Current Biology, Vol. 15, 1501-1507, Aug. 23, 2005, the contents of which are incorporated herein by reference in its entirety).

The polynucleotides may be designed to encode transcripts with alternative polyA tail structures including histone mRNA. These mRNAs are distinguished by their lack of a 3′ poly(A) tail, the function of which is instead assumed by a stable stem-loop structure and its cognate stem-loop binding protein (SLBP); the latter carries out the same functions as those of PABP on polyadenylated mRNAs

Unique poly-A tail lengths provide certain advantages to the polynucleotides of the present invention (e.g., antigen-encoding polynucleotides featured in the NAVs of the invention).

Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1.500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1.000 to 3,000, from 1,500 to 2,000, from 1,500 to 2,500, from 1,500 to 3,000, from 2,000 to 3,000, from 2,000 to 2,500, and from 2,500 to 3,000).

In one embodiment, the poly-A tail is designed relative to the length of the overall polynucleotide or the length of a particular region of the polynucleotide. This design may be based on the length of a coding region, the length of a particular feature or region or based on the length of the ultimate product expressed from the polynucleotides.

In this context the poly-A tail may be 10, 20, 30, 40, 50, 60, 70, 80, 90, or 100% greater in length than the polynucleotide or feature thereof. The poly-A tail may also be designed as a fraction of the polynucleotides to which it belongs. In this context, the poly-A tail may be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein may enhance expression.

Start Codon Region. In some aspects, the polynucleotides may have regions that are analogous to or function like a start codon region.

In one embodiment, the translation of a polynucleotide may initiate on a codon which is not the start codon AUG. Translation of the polynucleotide may initiate on an alternative start codon such as, but not limited to, ACG, AGG, AAG, CTG/CUG, GTG/GUG, ATA/AUA, ATT/AUU, TTG/UUG. As a non-limiting example, the translation of a polynucleotide begins on the alternative start codon ACG. As another non-limiting example, polynucleotide translation begins on the alternative start codon CTG or CUG. As yet another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See e.g., Matsuda and Mauro PLoS ONE, 2010 5:11; the contents of which are herein incorporated by reference in its entirety). Masking any of the nucleotides flanking a codon that initiates translation may be used to alter the position of translation initiation, translation efficiency, length and/or structure of a polynucleotide.

Stop Codon Region. In one aspect, the polynucleotides may include at least one or two stop codons before the 3′ untranslated region (UTR). The stop codon may be selected from TGA, TAA and TAG. In one aspect, the polynucleotides include the stop codon TGA and one additional stop codon. In a further embodiment the addition stop codon may be TAA. In another embodiment, the polynucleotides of the present invention include three stop codons.

Signal Sequences. The polynucleotides described herein may also encode additional features which facilitate trafficking of the polypeptides to therapeutically relevant sites. One such feature which aids in protein trafficking is the signal sequence. As used herein, a “signal sequence” or “signal peptide” is a polynucleotide or polypeptide, respectively, which is from about 9 to 200 nucleotides (3-60 amino acids) in length which is incorporated at the 5′ (or N-terminus) of the coding region or polypeptide encoded, respectively. Addition of these sequences result in trafficking of the encoded polypeptide to the endoplasmic reticulum through one or more secretory pathways. Some signal peptides are cleaved from the protein by signal peptidase after the proteins are transported.

Protein Cleavage Signals and Sites. In certain aspects, polypeptides of the invention (e.g., antigen polypeptides) may include various protein cleavage signals and/or sites.

In one embodiment, the polypeptides of the present invention may include at least one protein cleavage signal containing at least one protein cleavage site. The protein cleavage site may be located at the N-terminus, the C-terminus, at any space between the N- and the C-termini such as, but not limited to, half-way between the N- and C-termini, between the N-terminus and the half-way point, between the half-way point and the C-terminus, and combinations thereof.

In one embodiment, the polynucleotides of the present invention may be engineered such that the polynucleotide contains at least one encoded protein cleavage signal. The encoded protein cleavage signal may be located in any region including but not limited to before the start codon, after the start codon, before the coding region, within the coding region such as, but not limited to, half way in the coding region, between the start codon and the half way point, between the half way point and the stop codon, after the coding region, before the stop codon, between two stop codons, after the stop codon and combinations thereof.

In one embodiment, the polynucleotides of the present invention may include at least one encoded protein cleavage signal containing at least one protein cleavage site. The encoded protein cleavage signal may include, but is not limited to, signalase cleavage signal (SEQ ID NO:10), a proprotein convertase (or prohormone convertase), thrombin and/or Factor Xa protein cleavage signal.

Codon Optimization. The polynucleotides contained in the NAVs of the invention, their regions or parts or subregions may be codon optimized. Codon optimization methods are known in the art and may be useful in efforts to achieve one or more of several goals. These goals include to match codon frequencies in target and host organisms to ensure proper folding, bias GC content to increase mRNA stability or reduce secondary structures, minimize tandem repeat codons or base runs that may impair gene construction or expression, customize transcriptional and translational control regions, insert or remove protein trafficking sequences, remove/add post translation modification sites in encoded protein (e.g. glycosylation sites), add, remove or shuffle protein domains, insert or delete restriction sites, modify ribosome binding sites and mRNA degradation sites, to adjust translational rates to allow the various domains of the protein to fold properly, or to reduce or eliminate problem secondary structures within the polynucleotide. Codon optimization tools, algorithms and services are known in the art, non-limiting examples include services from GeneArt (Life Technologies), DNA2.0 (Menlo Park Calif.) and/or proprietary methods. In one embodiment, the ORF sequence is optimized using optimization algorithms.

In some embodiments, a 5′ UTR and/or a 3′ UTR region may be provided as flanking regions. Multiple 5′ or 3′ UTRs may be included in the flanking regions and may be the same or of different sequences. Any portion of the flanking regions, including none, may be codon optimized and any may independently contain one or more different structural or chemical modifications, before and/or after codon optimization.

In Vitro Transcription-Enzymatic Synthesis. cDNA encoding the polynucleotides described herein may be transcribed using an in vitro transcription (IVT) system. The system typically comprises a transcription buffer, nucleotide triphosphates (NTPs), an RNase inhibitor and a polymerase. The NTPs may be manufactured in house, may be selected from a supplier, or may be synthesized as described herein. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate polynucleotides (e.g., modified nucleic acids).

Solid-Phase Chemical Synthesis. Chimeric polynucleotides or circular polynucleotides described herein may be manufactured in whole or in part using solid phase techniques.

Solid-phase chemical synthesis of polynucleotides or nucleic acids is an automated method wherein molecules are immobilized on a solid support and synthesized step by step in a reactant solution. Impurities and excess reagents are washed away and no purification is required after each step. The automation of the process is amenable on a computer-controlled solid-phase synthesizer. Solid-phase synthesis allows rapid production of polynucleotides or nucleic acids in a relatively large scale that leads to the commercial availability of some polynucleotides or nucleic acids. Furthermore, it is useful in site-specific introduction of chemical modifications in the polynucleotide or nucleic acid sequences. It is an indispensable tool in designing modified derivatives of natural nucleic acids.

Liquid Phase Chemical Synthesis. The synthesis of chimeric polynucleotides or circular polynucleotides of the present invention (e.g., antigen-encoding polynucleotides featured in the NAVs of the invention) by the sequential addition of monomer building blocks may be carried out in a liquid phase. A covalent bond is formed between the monomers or between a terminal functional group of the growing chain and an incoming monomer. Functional groups not involved in the reaction must be temporarily protected. After the addition of each monomer building block, the reaction mixture has to be purified before adding the next monomer building block. The functional group at one terminal of the chain has to be deprotected to be able to react with the next monomer building blocks. A liquid phase synthesis is labor- and time-consuming and cannot not be automated. Despite the limitations, liquid phase synthesis is still useful in preparing short polynucleotides in a large scale. Because the system is homogenous, it does not require a large excess of reagents and is cost-effective in this respect.

Combination of Synthetic Methods. The synthetic methods discussed above each has its own advantages and limitations. Attempts have been conducted to combine these methods to overcome the limitations. Such combinations of methods are within the scope of the present invention.

### III. MODIFICATIONS

In certain embodiments, polynucleotides described herein can include various substitutions and/or insertions. As used herein the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population. Generally, herein, these terms are not intended to refer to the ribonucleotide modifications in naturally occurring 5′-terminal mRNA cap moieties. In a polypeptide, the term “modification” refers to a modification as compared to the canonical set of 20 amino acids.

The modifications may be various distinct modifications. In some embodiments, the regions may contain one, two, or more (optionally different) nucleoside or nucleotide modifications. In some embodiments, a modified polynucleotide, introduced to a cell may exhibit reduced degradation in the cell, as compared to an unmodified polynucleotide.

The polynucleotides of the NAVs of the invention can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

Non-natural modified nucleotides may be introduced to polynucleotides during synthesis or post-synthesis of the chains to achieve desired functions or properties. The modifications may be on internucleotide lineage, the purine or pyrimidine bases, or sugar. The modification may be introduced at the terminal of a chain or anywhere else in the chain; with chemical synthesis or with a polymerase enzyme.

Modified Polynucleotide Molecules. The present invention also includes building blocks, e.g., modified ribonucleosides, and modified ribonucleotides, of polynucleotide molecules, e.g., of the NAVs of the invention. For example, these building blocks can be useful for preparing the polynucleotides of the invention.

Modifications on the Sugar. The modified nucleosides and nucleotides which may be incorporated into a polynucleotide can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C1-6 alkyl: optionally substituted C1-6 alkoxy; optionally substituted C6-10 aryloxy; optionally substituted C3-8 cycloalkyl; optionally substituted C3-8 cycloalkoxy; optionally substituted C6-10 aryloxy; optionally substituted C6-10 aryl-C1-6 alkoxy, optionally substituted C1-12 (heterocyclyl)oxy; a sugar (e.g., ribose, pentose, or any described herein); a polyethyleneglycol (PEG), O(CH₂CH₂O)nCH₂CH₂R, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C1-6 alkylene or C1-6 heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein

Generally, RNA includes the sugar group ribose, which is a 5-membered ring having an oxygen. Exemplary, non-limiting modified nucleotides include replacement of the oxygen in ribose (e.g., with S, Se. or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar. Such sugar modifications are taught International Application Number PCT/2012/058519 filed Oct. 3, 2012 (Attorney Docket Number M9) and U.S. Provisional Application No. 61/837,297 filed Jun. 20, 2013 (Attorney Docket Number M36) the contents of each of which are incorporated herein by reference in its entirety.

Modifications on the Nucleobase. As described herein “nucleoside” is defined as a compound containing a sugar molecule (e.g., a pentose or ribose) or a derivative thereof in combination with an organic base (e.g., a purine or pyrimidine) or a derivative thereof (also referred to herein as “nucleobase”). As described herein, “nucleotide” is defined as a nucleoside including a phosphate group. The modified nucleotides may by synthesized by any useful method, as described herein (e.g., chemically, enzymatically, or recombinantly to include one or more modified or non-natural nucleosides). The polynucleotides may comprise a region or regions of linked nucleosides. Such regions may have variable backbone linkages. The linkages may be standard phosphoester linkages, in which case the polynucleotides would comprise regions of nucleotides.

The modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil.

The modified nucleosides and nucleotides can include a modified nucleobase. Examples of nucleobases found in RNA include, but are not limited to, adenine, guanine, cytosine, and uracil. Examples of nucleobase found in DNA include, but are not limited to, adenine, guanine, cytosine, and thymine. Such modified nucleobases (including the distinctions between naturally occurring and non-naturally occurring) are taught in International Application Number PCT/2012/058519 filed Oct. 3, 2012 (Attorney Docket Number M9) and U.S. Provisional Application No. 61/837,297 filed Jun. 20, 2013 (Attorney Docket Number M36) the contents of each of which are incorporated herein by reference in its entirety.

### IV. PHARMACEUTICAL VACCINE COMPOSITIONS

The present invention provides pharmaceutical compositions including NAVs and NAV compositions and/or complexes optionally in combination with one or more pharmaceutically acceptable excipients. The present invention provides NAVs and NAV pharmaceutical compositions and complexes optionally in combination with one or more pharmaceutically acceptable excipients. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21′ ed., Lippincott Williams & Wilkins, 2005 (incorporated herein by reference in its entirety).

In some embodiments, compositions are administered to humans, human patients or subjects. For the purposes of the present disclosure, the phrase “active ingredient” generally refers to the NAVs or the polynucleotides contained therein, e.g., antigen-encoding polynucleotides, for example, RNA polynucleotides, to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%. e.g., between 0.5 and 50%, between 1-30%, between 5-80%, at least 80% (w/w) active ingredient.

Formulations. The NAVs of the invention can be formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with NAVs (e.g., for transplantation into a subject), hyaluronidase, nanoparticle mimics and combinations thereof.

Accordingly, the formulations of the invention can include one or more excipients, each in an amount that may increases the stability of the NAV, increases cell transfection by the NAV, increases the expression of polynucleotides encoded protein, and/or alters the release profile of polynucleotide encoded proteins. Further, the polynucleotides of the present invention may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the formulations described herein may contain at least one polynucleotide, e.g., antigen-encoding polynucleotide. As a non-limiting example, the formulations may contain 1, 2, 3, 4 or 5 polynucleotides.

In one embodiment, the formulations described herein may comprise more than one type of polynucleotide, e.g., antigen-encoding polynucleotide. In one embodiment, the formulation may comprise a chimeric polynucleotide in linear and circular form. In another embodiment, the formulation may comprise a circular polynucleotide and an IVT polynucleotide. In yet another embodiment, the formulation may comprise an IVT polynucleotide, a chimeric polynucleotide and a circular polynucleotide.

In one embodiment the formulation may contain polynucleotide encoding proteins selected from categories such as, but not limited to, human proteins, veterinary proteins, bacterial proteins, biological proteins, antibodies, immunogenic proteins, therapeutic peptides and proteins, secreted proteins, plasma membrane proteins, cytoplasmic and cytoskeletal proteins, intracellular membrane bound proteins, nuclear proteins, proteins associated with human disease and/or proteins associated with non-human diseases. In one embodiment, the formulation contains at least three polynucleotides encoding proteins. In one embodiment, the formulation contains at least five polynucleotide encoding proteins.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In some embodiments, the particle size of the lipid nanoparticle may be increased and/or decreased. The change in particle size may be able to help counter biological reaction such as, but not limited to, inflammation or may increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients may optionally be included in the pharmaceutical formulations of the invention.

### Liposomes, Lipoplexes, and Lipid Nanoparticles

The NAVs of the invention can be formulated using one or more liposomes, lipoplexes, or lipid nanoparticles. In one embodiment, pharmaceutical compositions of NAVs include liposomes. Liposomes are artificially prepared vesicles which may primarily be composed of a lipid bilayer and may be used as a delivery vehicle for the administration of nutrients and pharmaceutical formulations. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

In one embodiment, pharmaceutical compositions described herein may include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120; herein incorporated by reference in its entirety) and liposomes which may deliver small molecule drugs such as, but not limited to, DOXIL® from Janssen Biotech, Inc. (Horsham, Pa.).

As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

Peptides and Proteins. The NAVs of the invention can be formulated with peptides and/or proteins in order to increase transfection of cells by the polynucleotide. In one embodiment, peptides such as, but not limited to, cell penetrating peptides and proteins and peptides that enable intracellular delivery may be used to deliver pharmaceutical formulations. A non-limiting example of a cell penetrating peptide which may be used with the pharmaceutical formulations of the present invention includes a cell-penetrating peptide sequence attached to polycations that facilitates delivery to the intracellular space, e.g., HIV-derived TAT peptide, penetratins, transportans, or hCT derived cell-penetrating peptides (see, e.g., Caron et al., Mol. Ther. 3(3):310-8 (2001); Langel, Cell-Penetrating Peptides: Processes and Applications (CRC Press, Boca Raton Fla., 2002); El-Andaloussi et al., Curr. Pharm. Des. 1(28):3597-611 (2003); and Deshayes et al., Cell. Mol. Life Sci. 62(16):1839-49 (2005), all of which are incorporated herein by reference in their entirety). The compositions can also be formulated to include a cell penetrating agent, e.g., liposomes, which enhance delivery of the compositions to the intracellular space. NAVs of the invention may be complexed to peptides and/or proteins such as, but not limited to, peptides and/or proteins from Aileron Therapeutics (Cambridge, Mass.) and Permeon Biologics (Cambridge, Mass.) in order to enable intracellular delivery (Cronican et al., ACS Chem. Biol. 2010 5:747-752; McNaughton et al., Proc. Natl. Acad. Sci. USA 2009 106:6111-6116; Sawyer, Chem Biol Drug Des. 2009 73:3-6; Verdine and Hilinski, Methods Enzymol. 2012; 503:3-33; all of which are herein incorporated by reference in its entirety).

Cells. The NAVs of the invention can be transfected ex vivo into cells, which are subsequently transplanted into a subject. As one non-limiting example, a sample of blood from a patient or subject may be treated with an antigen or adjuvant or both where one or more are encoded by the NAVs of the invention to activate the PBMC population. This activated sample or a subset of specific cells may then be given back to the donor patient thereby activating the immune system. This activated PBMC vaccine may be designed using any of the NAVs of the present disclosure. As non-limiting examples, the pharmaceutical compositions may include red blood cells to deliver modified RNA to liver and myeloid cells, virosomes to deliver modified RNA in virus-like particles (VLPs), and electroporated cells such as, but not limited to, from MAXCYTE® (Gaithersburg, Md.) and from ERYTECH® (Lyon, France) to deliver modified RNA. Examples of use of red blood cells, viral particles and electroporated cells to deliver payloads other than polynucleotides have been documented (Godfrin et al., Expert Opin Biol Ther. 2012 12:127-133; Fang et al., Expert Opin Biol Ther. 2012 12:385-389; Hu et al., Proc Nat Acad Sci USA. 2011 108:10980-10985; Lund et al., Pharm Res. 2010 27:400-420; Huckriede et al., J Liposome Res. 2007; 17:39-47; Cusi, Hum Vaccin. 2006 2:1-7; de Jonge et al., Gene Ther. 2006 13:400-411; all of which are herein incorporated by reference in its entirety).

Suspension Formulations. In some embodiments, suspension formulations are provided comprising NAVs, water immiscible oil depots, surfactants and/or co-surfactants and/or co-solvents. Combinations of oils and surfactants may enable suspension formulation with NAVs. Delivery of NAVs in a water immiscible depot may be used to improve bioavailability through sustained release of NAVs from the depot to the surrounding physiologic environment and prevent polynucleotides degradation by nucleases.

In some embodiments, suspension formulations of NAV may be prepared using combinations of polynucleotides, oil-based solutions and surfactants. Such formulations may be prepared as a two-part system comprising an aqueous phase comprising polynucleotides and an oil-based phase comprising oil and surfactants. Exemplary oils for suspension formulations may include, but are not limited to sesame oil and Miglyol (comprising esters of saturated coconut and palmkernel oil-derived caprylic and capric fatty acids and glycerin or propylene glycol), corn oil, soybean oil, peanut oil, beeswax and/or palm seed oil. Exemplary surfactants may include, but are not limited to Cremophor, polysorbate 20, polysorbate 80, polyethylene glycol, transcutol, Capmul®, labrasol, isopropyl myristate, and/or Span 80. In some embodiments, suspensions may comprise co-solvents including, but not limited to ethanol, glycerol and/or propylene glycol.

Excipients. NAV pharmaceutical formulations may additionally comprise a pharmaceutically acceptable excipient, which, as used herein, includes, but are not limited to, any and all solvents, dispersion media, diluents, or other liquid vehicles, dispersion or suspension aids, surface active agents, isotonic agents, thickening or emulsifying agents, preservatives, solid binders, lubricants, flavoring agents, stabilizers, antioxidants, osmolality adjusting agents. pH adjusting agents and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro (Lippincott, Williams & Wilkins, Baltimore, Md., 2006; incorporated herein by reference in its entirety). The use of a conventional excipient medium may be contemplated within the scope of the present disclosure, except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions. The composition may also include excipients such as cocoa butter and suppository waxes, coloring agents, coating agents, sweetening, flavoring, and/or perfuming agents.

Cryoprotectants. In some embodiments, NAV formulations may comprise cyroprotectants. As used herein, there term “cryoprotectant” refers to one or more agent that when combined with a given substance, helps to reduce or eliminate damage to that substance that occurs upon freezing. In some embodiments, cryoprotectants are combined with NAVs in order to stabilize them during freezing. Frozen storage of NAVs between −20° C. and −80° C. may be advantageous for long term (e.g. 36 months) stability of polynucleotide. In some embodiments, cryoprotectants are included in NAV formulations to stabilize polynucleotide through freeze/thaw cycles and under frozen storage conditions. Cryoprotectants of the present invention may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

Bulking Agents. In some embodiments, NAV formulations may comprise bulking agents. As used herein, there term “bulking agent” refers to one or more agents included in formulations to impart a desired consistency to the formulation and/or stabilization of formulation components. In some embodiments, bulking agents are included in lyophilized NAV formulations to yield a “pharmaceutically elegant” cake, stabilizing the lyophilized NAVs during long term (e.g. 36 month) storage. Bulking agents of the present invention may include, but are not limited to sucrose, trehalose, mannitol, glycine, lactose and/or raffinose. In some embodiments, combinations of cryoprotectants and bulking agents (for example, sucrose/glycine or trehalose/mannitol) may be included to both stabilize NAVs during freezing and provide a bulking agent for lyophilization.

Administration. The NAVs of the present invention may be administered by any route which results in a therapeutically effective outcome.

### Parenteral and Injectable Administration

Liquid dosage forms for parenteral administration include, but are not limited to, pharmaceutically acceptable emulsions, microemulsions, solutions, suspensions, syrups, and/or elixirs.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

Dosing. The present invention provides methods comprising administering NAVs and in accordance with the invention to a subject in need thereof. The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect (see e.g., the range of unit doses described in International Publication No WO2013078199, herein incorporated by reference in its entirety). The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

According to the present invention, NAVs may be administered in split-dose regimens. As used herein, a “split dose” is the division of single unit dose or total daily dose into two or more doses, e.g., two or more administrations of the single unit dose. As used herein, a “single unit dose” is a dose of any therapeutic administer in one dose/at one time/single route/single point of contact, i.e., single administration event. As used herein, a “total daily dose” is an amount given or prescribed in 24 hr period. It may be administered as a single unit dose. In one embodiment, the NAVs of the present invention are administer to a subject in split doses. The NAVs may be formulated in buffer only or in a formulation described herein.

### Multi-Dose and Repeat-Dose Administration

In some embodiments, NAV compounds and/or compositions of the present invention may be administered in two or more doses (referred to herein as “multi-dose administration”). Such doses may comprise the same components or may comprise components not included in a previous dose. Such doses may comprise the same mass and/or volume of components or an altered mass and/or volume of components in comparison to a previous dose. In some embodiments, multi-dose administration may comprise repeat-dose administration. As used herein, the term “repeat-dose administration” refers to two or more doses administered consecutively or within a regimen of repeat doses comprising substantially the same components provided at substantially the same mass and/or volume. In some embodiments, subjects may display a repeat-dose response. As used herein, the term “repeat-dose response” refers to a response in a subject to a repeat-dose that differs from that of another dose administered within a repeat-dose administration regimen. In some embodiments, such a response may be the expression of a protein in response to a repeat-dose comprising NAV. In such embodiments, protein expression may be elevated in comparison to another dose administered within a repeat-dose administration regimen or protein expression may be reduced in comparison to another dose administered within a repeat-dose administration regimen. Alteration of protein expression may be from about 1% to about 20%, from about 5% to about 50% from about 10% to about 60%, from about 25% to about 75%, from about 40% to about 100% and/or at least 100%. A reduction in expression of mRNA administered as part of a repeat-dose regimen, wherein the level of protein translated from the administered RNA is reduced by more than 40% in comparison to another dose within the repeat-dose regimen is referred to herein as “repeat-dose resistance.”

### V. KITS AND DEVICES

The invention provides a variety of kits for conveniently and/or effectively carrying out methods of the present invention. Typically, kits will comprise sufficient amounts and/or numbers of components to allow a user to perform multiple treatments of a subject(s) and/or to perform multiple experiments.

In one aspect, the present invention provides kits comprising the NAV molecules (including any proteins or polynucleotides) of the invention. In one embodiment, the kit comprises one or more functional antigens or function fragments thereof.

The kits can be for protein production, comprising a first polynucleotides comprising a translatable region of an antigen. The kit may further comprise packaging and instructions and/or a delivery agent to form a formulation composition. The delivery agent may comprise a saline, a buffered solution, or a delivery agent.

In one embodiment, the buffer solution may include sodium chloride, calcium chloride, phosphate and/or EDTA. In another embodiment, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose. In a further embodiment, the buffer solutions may be precipitated or it may be lyophilized. The amount of each component may be varied to enable consistent, reproducible higher concentration saline or simple buffer formulations. The components may also be varied in order to increase the stability of polynucleotides in the buffer solution over a period of time and/or under a variety of conditions.

In one aspect, the present invention provides kits for protein production, comprising: a polynucleotide comprising a translatable region, provided in an amount effective to produce a desired amount of a protein encoded by the translatable region when introduced into a target cell.

Devices. The present invention provides for devices which may incorporate RNAVs comprising polynucleotides that encode polypeptides of interest, e.g., encode antigenic polypeptides. These devices contain in a stable formulation the reagents to synthesize a polynucleotide in a formulation available to be immediately delivered to a subject in need thereof, such as a human patient.

Devices for administration may be employed to deliver the NAVs of the present invention according to single, multi- or split-dosing regimens taught herein.

Method and devices known in the art for multi-administration to cells, organs and tissues are contemplated for use in conjunction with the methods and compositions disclosed herein as embodiments of the present invention. These include, for example, those methods and devices having multiple needles, hybrid devices employing for example lumens or catheters as well as devices utilizing heat, electric current or radiation driven mechanisms.

In one embodiment, the NAV is administered subcutaneously or intramuscularly via at least 3 needles to three different, optionally adjacent, sites simultaneously, or within a 60 minutes period (e.g., administration to 4, 5, 6, 7, 8, 9, or 10 sites simultaneously or within a 60 minute period).

### VI. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Monocistronic linear Andes virus mRNA vaccine demonstrates protection of Syrian hamsters from lethal challenge with Andes virus. Syrian hamsters were immunized intramuscularly twice, on days 0 and 21, with 5 μg or 25 μg of Andes virus mRNA vaccine comprised of regular (non-modified uridine) or modified (N1-methylpseudouridine) construct, five animals in each group. Twenty one days after the second (booster) vaccination, the animals were challenged intramuscularly with 200 plaque forming units (PFU) of Andes virus. All five control animals developed severe clinical signs and were euthanized on 9 days post infection (dpi), as well two of the five animals vaccinate with 5 μg of modified mRNA vaccine construct. Conversely, all animals vaccinated with 5 μg or 25 μg of the regular, all five animals vaccinated with 25 μg of modified vaccine construct, and the remaining three animals vaccinated with 3 μg of modified vaccine construct survived the challenge without any clinical signs.

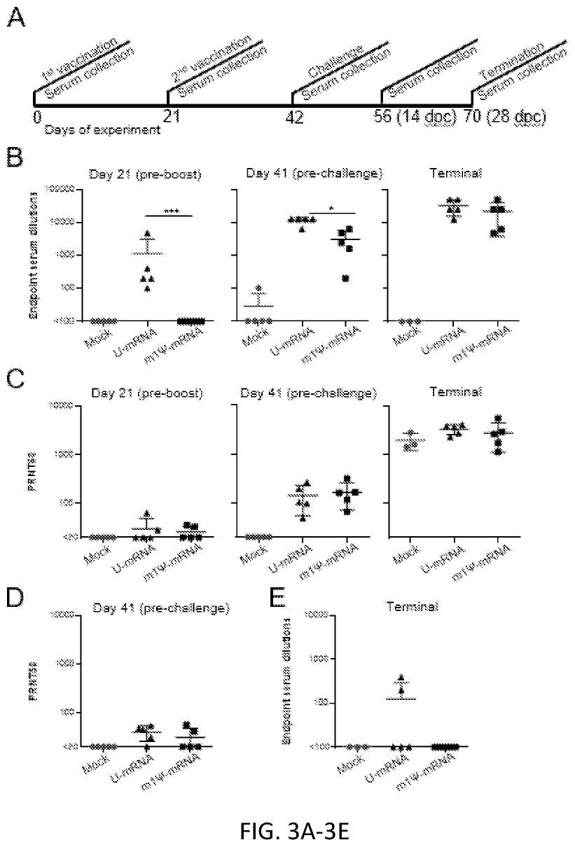

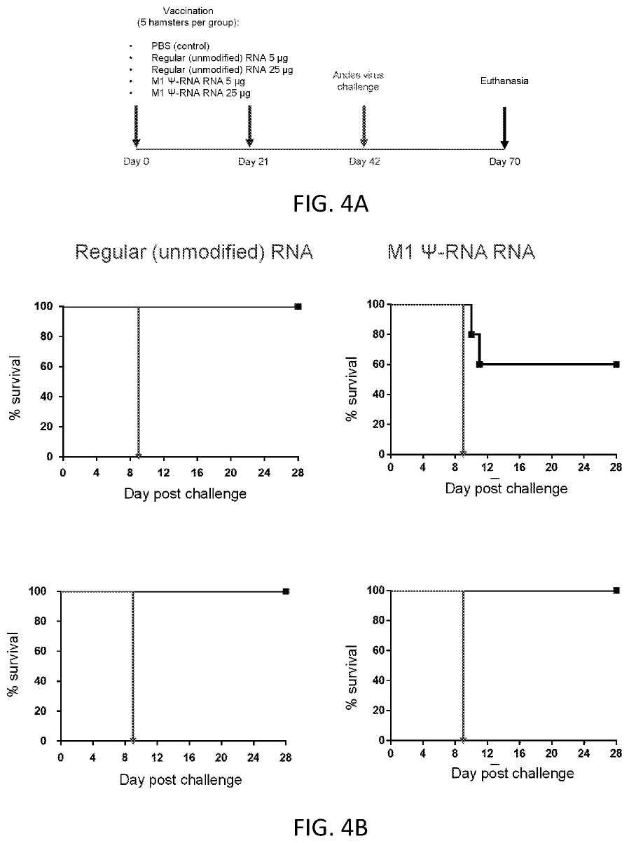

Monocistronic linear Andes virus mRNA vaccine elicits development of Andes virus neutralizing antibodies in Syrian hamsters (see for example FIG. 3 and FIG. 4). Syrian hamsters were immunized intramuscularly twice, on days 0 and 21, with 5 μg or 25 μg of Andes virus mRNA vaccine comprised of regular (non-modified uridine) or modified (N1-methylpseudouridine) construct, five animals in each group. Blood serum was taken from the animals on days 21 (prior to the second vaccination), 41 (20 days after the second vaccination, one day prior to challenge) and tested against ˜50 PFU of Andes virus in a standard plaque reduction neutralization test (PRNT). Limited amounts of neutralizing antibodies were determined in 20-80% of animals in each group on day 21 after first immunization. However, greater neutralizing antibody titers were determined in all but two animals on day 41 (20 days after the second vaccination). The two animals that did not demonstrate virus-neutralizing antibodies at this latter time point were vaccinated with 5 μg of modified Andes mRNA vaccine construct. FIG. 4B illustrates the survival of Syrian hamsters vaccinated intramuscularly with regular (non-modified uridine) or modified (N1-pseudouridine) monocistronic linear Andes vaccine constructs and challenged intramuscularly with Andes virus.

1. A Hantavirus vaccine, comprising an engineered messenger ribonucleic acid (mRNA) comprising an open reading frame encoding an antigenic Gn, Gc, or Gn and Gc protein.

2. The vaccine of claim 1, wherein the Gn and Gc proteins are encoded as a polyprotein.

3. The vaccine of claim 2, wherein the encoded polyprotein comprises a protease cleavage site between the Gn and Gc proteins.

4. The vaccine of claim 1, wherein the Gn and Gc proteins are encoded by separate open reading frames (ORF).

5. The vaccine of claim 4, wherein the Gn ORF and the Gc ORF are separated by an internal ribosome entry site (IRES).

6. The vaccine of claim 1, wherein the mRNA is linear.

7. The vaccine of claim 6, further comprising a 5′ UTR.

8. The vaccine of any one of claim 6 or 7, further comprising a 3′ UTR.

9. The vaccine of any one of claim 6, 7, or 8, further comprising a polyadenylation segment.

10. The vaccine of claim 1, wherein the mRNA is circular.

11. The vaccine of claim 10, further comprising 5′ region comprising from 5′ to 3′ (i) a 5′ external homology segment, (ii) a 3′ intron and exon segment, (iii) a 5′ internal homology segment, and (iv) a poly adenosine/cytosine spacer.

12. The vaccine of claim 10, further comprising 3′ region comprising from 5′ to 3′ (i) a poly adenosine/cytosine spacer, (ii) a 3′ internal homology segment, (iii) a 5′ intron and exon segment, and (iv) a 3′ external homology segment.

13. The vaccine of claim 10, wherein the Gn and Gc proteins are encoded as a polyprotein.

14. The vaccine of claim 13, wherein the encoded polyprotein comprises a protease cleavage site between the Gn and Gc proteins.

15. The vaccine of claim 10, wherein the Gn and Gc proteins are encoded by separate open reading frames (ORF).

16. The vaccine of claim 15, wherein the Gn ORF and the Gc ORF are separated by an internal ribosome entry site (IRES).

17. The vaccine of claim 1, wherein the vaccine has a nucleotide sequence that is 80, 85, 90, 95, 98, 99, 100% identical to SEQ ID NO: 13, SEQ ID NO:14, SEQ ID NO:15, SEQ ID NO:16, SEQ ID NO:17, or SEQ ID NO: 18.

18. A DNA construct encoding the vaccine of claim 1.

19. A method of inducing an antigen-specific immune response in a subject, the method comprising administering to the subject the vaccine of any one of claims 1 to 17 to produce an antigen-specific immune response in the subject.

20. A composition comprising a messenger ribonucleic acid (mRNA) of any one of claims 1 to 17 in a lipid particle.


SEQUENCE LISTING (18 sequences)

SEQ ID NO: 1 - DNA, 3942 bp, synthetic construct
  misc_feature 1..3942: note = artificial polynucleotide
  source 1..3942: mol_type = other DNA
acgcggccgctaatacgactcactataaggctagtagtagactccgcaagaagaagcaaaaaattaaagaagtgagtttaaaatggaaggctggtacctggtcgtgctgggcgtgtgttacaccctaacactggccatgcccaagaccatctacgagctgaagatggaatgccctcacaccgtgggcctcggccagggatatatcattggcagcaccgagctgggcctgatcagcatcgaggctgcctccgacatcaagctggaatctagctgcaacttcgacctgcacaccacatccatggctcaaaaaagcttcacccaggtggaatggcggaagaagagcgacaccacagacacaactaacgctgccagcacaacctttgaagcccagaccaaaactgtgaatctgcggggcacctgtatcctggctcctgaactgtacgatacactgaagaaggtgaagaaaacagtgctttgttatgacctgacctgcaaccagacacactgccagcctacagtgtacctgatcgcccctgtgctcacatgcatgagcatcagaagttgcatggccagcgtgttcacaagcagaatccaggtgatctacgagaagacacactgcgtgaccggacagctcatcgagggccaatgtttcaaccccgcccacaccctgaccctgagccagcctgcccacacctacgacaccgtgacactgcctataagctgcttcttcaccccaaagaagagcgaacagctgaaggtgattaagacattcgagggcatcctgactaagaccggctgcacagaaaacgccctgcagggctactacgtgtgcttcctgggcagccactctgagcccctgatcgtgccctctctggaagacatcagatccgccgaagtggtatctagaatgcttgtgcacccccgtggcgaggatcacgatgccatccagaactcacagagccacctgcggatcgtgggccctatcaccgccaaggtgccttctaccagcagcaccgacaccctgaagggcacagcatttgccggcgtgccaatgtacagcagtctgagcacactggtcagaaacgcggaccctgaattcgtgttcagccccggcatcgtgcctgagagcaaccacagcacctgtgacaagaagaccgtgccgatcacctggaccggctacctgcctatctccggcgaaatggaaaaagttacaggatgcacggtgttctgcaccctggccggccccggagccagctgcgaggcctactccgagaacggaatcttcaatatcagcagccctacctgcctggtgaacaaggtgcagcggttccggggcagcgagcagaagatcaacttcatctgccagagagtggaccaggacgtggtggtctactgcaatggacagaaaaaggtgatcctgaccaagacactggtgatcggccagtgtatctacacatttaccagcctgttctccctgatgcctgatgtggcccacagcctggctgttgagctgtgtgtgcctggcctgcacggctgggccacggtgatgctgttaagcaccttctgcttcggctgggtcctgatccccgctgtgacactgattatccttaagtgcctgagagtgctgacattttcttgtagccactacaccaacgagtccaagttcaagttcatcctggagaaggtgaagatcgagtaccagaagaccatgggcagcatggtgtgcgacgtgtgtcaccacgagtgcgagactgccaaggaactcgaatcccacagacagagctgcatcaacggccagtgtccttactgcatgaccatcaccgaggctacagagtctgccctgcaggcccattacagcatctgcaagctgaccggtagattccaggaggccctgaagaaatctctgaaaaagcctgaggtgaaaaagggctgttaccgcaccctgggcgtgttcagatacaagagcagatgttacgtgggcttggtgtggtgcctgctgctgacttgtgagatcgtgatctgggccgccagcgccgaaacacccctgatggaaagcgggtggagtgacaccgcccacggagtgggagagatccccatgaagaccgatctggagctggacttcagcctgcctagctcctcaagctacagctacagacggaagctgaccaaccccgctaacaaagaggaaagcatcccgttccacttccagatggaaaagcaagtgatccacgctgagatccagcctctgggacactggatggacgctaccttcaatatcaagaccgcctttcactgctacggcgcctgccagaaatattcttacccctggcagacctccaagtgttttttcgagaaagactaccagtacgagacaggatggggttgcaaccctggcgactgccctggagtgggcaccggatgcaccgcctgcggtgtgtacctagataagctgaagtctgttgggaaggcctacaagatcatttctctgaagtacacaagaaaggtgtgcatccagctgggcaccgagcaaacctgcaagcacatcgatgctaacgactgtctcgtcacccccagtgtgaaggtgtgcatcgtgggcacagtgagcaaacttcagcctagcgacaccctgctgttcctgggaccacttgagcagggcggaatcatcctgaagcagtggtgcaccacctcctgcgccttcggcgatcctggcgacatcatgagcaccccttctggcatgagatgcccagagcacacaggcagcttcagaaagatctgtggcttcgccactacccccgtgtgtgagtaccaaggcaacaccatctctggatataagcggatgatggccaccaaggacagcttccaaagcttcaacctgactgaacctcatataacaacaaacaagctggagtggatcgacccggatggcaacaccagagaccacgtgaacctggtgctgaacagagatgtctccttccaggatctgagcgataacccctgtaaagtggacctccatacccaggccatcgagggcgcttggggcagcggcgttggctttaccctgacatgtaccgtgggactgaccgaatgccctagctttatgacatctatcaaagcctgtgatctggccatgtgctacggcagcaccgtgactaacctggctagaggctctaataccgtcaaggtggtgggcaaaggcggccactccggctctagctttaagtgctgccacgacaccgactgcagcagcgagggcctgctggccagcgccccacatctggagcgggtgaccggcttcaaccagatcgacagcgacaaggtatacgacgacggtgcccctccttgcacatttaagtgctggttcaccaagtccggcgagtggctgctgggcatccttaacggcaattggatcgtggtggtggtgctggtggtgatcctgatcctgtcaatcattatgttcagcgtgctgtgccctcggagaggccacaagaaaaccgtctgacaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggtactgcatgcacgcaatgctagctgcccctttcccgtcctgggtaccccgagtctcccccgacctcgggtcccaggtatgctcccacctccacctgccccactcaccacctctgctagttccagacacctccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagatatcaaaaaaaaaaaaaaaaaaaaaaaaaaaaaattcgaaaaa

SEQ ID NO: 2 - protein, 1138 aa, synthetic construct
  REGION 1..1138: note = recombinant polypeptide
MEGWYLVVLGVCYTLTLAMPKTIYELKMECPHTVGLGQGYIIGSTELGLISIEAASDIKLESSCNFDLHTTSMAQKSFTQVEWRKKSDTTDTTNAASTTFEAQTKTVNLRGTCILAPELYDTLKKVKKTVLCYDLTCNQTHCQPTVYLIAPVLTCMSIRSCMASVFTSRIQVIYEKTHCVTGQLIEGQCFNPAHTLTLSQPAHTYDTVTLPISCFFTPKKSEQLKVIKTFEGILTKTGCTENALQGYYVCFLGSHSEPLIVPSLEDIRSAEVVSRMLVHPRGEDHDAIQNSQSHLRIVGPITAKVPSTSSTDTLKGTAFAGVPMYSSLSTLVRNADPEFVFSPGIVPESNHSTCDKKTVPITWTGYLPISGEMEKVTGCTVFCTLAGPGASCEAYSENGIFNISSPTCLVNKVQRFRGSEQKINFICQRVDQDVVVYCNGQKKVILTKTLVIGQCIYTFTSLFSLMPDVAHSLAVELCVPGLHGWATVMLLSTFCFGWVLIPAVTLIILKCLRVLTFSCSHYTNESKFKFILEKVKIEYQKTMGSMVCDVCHHECETAKELESHRQSCINGQCPYCMTITEATESALQAHYSICKLTGRFQEALKKSLKKPEVKKGCYRTLGVFRYKSRCYVGLVWCLLLTCEIVIWAASAETPLMESGWSDTAHGVGEIPMKTDLELDFSLPSSSSYSYRRKLTNPANKEESIPFHFQMEKQVIHAEIQPLGHWMDATFNIKTAFHCYGACQKYSYPWQTSKCFFEKDYQYETGWGCNPGDCPGVGTGCTACGVYLDKLKSVGKAYKIISLKYTRKVCIQLGTEQTCKHIDANDCLVTPSVKVCIVGTVSKLQPSDTLLFLGPLEQGGIILKQWCTTSCAFGDPGDIMSTPSGMRCPEHTGSFRKICGFATTPVCEYQGNTISGYKRMMATKDSFQSFNLTEPHITTNKLEWIDPDGNTRDHVNLVLNRDVSFQDLSDNPCKVDLHTQAIEGAWGSGVGFTLTCTVGLTECPSFMTSIKACDLAMCYGSTVTNLARGSNTVKVVGKGGHSGSSFKCCHDTDCSSEGLLASAPHLERVTGFNQIDSDKVYDDGAPPCTFKCWFTKSGEWLLGILNGNWIVVVVLVVILILSIIMFSVLCPRRGHKKTV

SEQ ID NO: 3 - DNA, 4758 bp, synthetic construct
  misc_feature 1..4758: note = artificial polynucleotide
  source 1..4758: mol_type = other DNA
acgcggccgctaatacgactcactataaggctagtagtagactccgcaagaagaagcaaaaaattaaagaagtgagtttaaaatggaaggctggtacctggtcgtgctgggcgtgtgttacaccctaacactggccatgcccaagaccatctacgagctgaagatggaatgccctcacaccgtgggcctcggccagggatatatcattggcagcaccgagctgggcctgatcagcatcgaggctgcctccgacatcaagctggaatctagctgcaacttcgacctgcacaccacatccatggctcaaaaaagcttcacccaggtggaatggcggaagaagagcgacaccacagacacaactaacgctgccagcacaacctttgaagcccagaccaaaactgtgaatctgcggggcacctgtatcctggctcctgaactgtacgatacactgaagaaggtgaagaaaacagtgctttgttatgacctgacctgcaaccagacacactgccagcctacagtgtacctgatcgcccctgtgctcacatgcatgagcatcagaagttgcatggccagcgtgttcacaagcagaatccaggtgatctacgagaagacacactgcgtgaccggacagctcatcgagggccaatgtttcaaccccgcccacaccctgaccctgagccagcctgcccacacctacgacaccgtgacactgcctataagctgcttcttcaccccaaagaagagcgaacagctgaaggtgattaagacattcgagggcatcctgactaagaccggctgcacagaaaacgccctgcagggctactacgtgtgcttcctgggcagccactctgagcccctgatcgtgccctctctggaagacatcagatccgccgaagtggtatctagaatgcttgtgcacccccgtggcgaggatcacgatgccatccagaactcacagagccacctgcggatcgtgggccctatcaccgccaaggtgccttctaccagcagcaccgacaccctgaagggcacagcatttgccggcgtgccaatgtacagcagtctgagcacactggtcagaaacgcggaccctgaattcgtgttcagccccggcatcgtgcctgagagcaaccacagcacctgtgacaagaagaccgtgccgatcacctggaccggctacctgcctatctccggcgaaatggaaaaagttacaggatgcacggtgttctgcaccctggccggccccggagccagctgcgaggcctactccgagaacggaatcttcaatatcagcagccctacctgcctggtgaacaaggtgcagcggttccggggcagcgagcagaagatcaacttcatctgccagagagtggaccaggacgtggtggtctactgcaatggacagaaaaaggtgatcctgaccaagacactggtgatcggccagtgtatctacacatttaccagcctgttctccctgatgcctgatgtggcccacagcctggctgttgagctgtgtgtgcctggcctgcacggctgggccacggtgatgctgttaagcaccttctgcttcggctgggtcctgatccccgctgtgacactgattatccttaagtgcctgagagtgctgacattttcttgtagccactacaccaacgagtccaagttcaagttcatcctggagaaggtgaagatcgagtaccagaagaccatgggcagcatggtgtgcgacgtgtgtcaccacgagtgcgagactgccaaggaactcgaatcccacagacagagctgcatcaacggccagtgtccttactgcatgaccatcaccgaggctacagagtctgccctgcaggcccattacagcatctgcaagctgaccggtagattccaggaggccctgaagaaatctctgaaaaagcctgaggtgaaaaagggctgttaccgcaccctgggcgtgttcagatacaagagcagatgttacgtgggcttggtgtggtgcctgctgctgacttgtgagatcgtgatctgggccgccagcgcctgattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgaaacacccctgatggaaagcgggtggagtgacaccgcccacggagtgggagagatccccatgaagaccgatctggagctggacttcagcctgcctagctcctcaagctacagctacagacggaagctgaccaaccccgctaacaaagaggaaagcatcccgttccacttccagatggaaaagcaagtgatccacgctgagatccagcctctgggacactggatggacgctaccttcaatatcaagaccgcctttcactgctacggcgcctgccagaaatattcttacccctggcagacctccaagtgttttttcgagaaagactaccagtacgagacaggatggggttgcaaccctggcgactgccctggagtgggcaccggatgcaccgcctgcggtgtgtacctagataagctgaagtctgttgggaaggcctacaagatcatttctctgaagtacacaagaaaggtgtgcatccagctgggcaccgagcaaacctgcaagcacatcgatgctaacgactgtctcgtcacccccagtgtgaaggtgtgcatcgtgggcacagtgagcaaacttcagcctagcgacaccctgctgttcctgggaccacttgagcagggcggaatcatcctgaagcagtggtgcaccacctcctgcgccttcggcgatcctggcgacatcatgagcaccccttctggcatgagatgcccagagcacacaggcagcttcagaaagatctgtggcttcgccactacccccgtgtgtgagtaccaaggcaacaccatctctggatataagcggatgatggccaccaaggacagcttccaaagcttcaacctgactgaacctcatataacaacaaacaagctggagtggatcgacccggatggcaacaccagagaccacgtgaacctggtgctgaacagagatgtctccttccaggatctgagcgataacccctgtaaagtggacctccatacccaggccatcgagggcgcttggggcagcggcgttggctttaccctgacatgtaccgtgggactgaccgaatgccctagctttatgacatctatcaaagcctgtgatctggccatgtgctacggcagcaccgtgactaacctggctagaggctctaataccgtcaaggtggtgggcaaaggcggccactccggctctagctttaagtgctgccacgacaccgactgcagcagcgagggcctgctggccagcgccccacatctggagcgggtgaccggcttcaaccagatcgacagcgacaaggtatacgacgacggtgcccctccttgcacatttaagtgctggttcaccaagtccggcgagtggctgctgggcatccttaacggcaattggatcgtggtggtggtgctggtggtgatcctgatcctgtcaatcattatgttcagcgtgctgtgccctcggagaggccacaagaaaaccgtctgacaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggtactgcatgcacgcaatgctagctgcccctttcccgtcctgggtaccccgagtctcccccgacctcgggtcccaggtatgctcccacctccacctgccccactcaccacctctgctagttccagacacctccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagatatcaaaaaaaaaaaaaaaaaaaaaaaaaaaaaattcgaaaaa

SEQ ID NO: 4 - protein, 651 aa, synthetic construct
  REGION 1..651: note = recombinant polypeptide
MEGWYLVVLGVCYTLTLAMPKTIYELKMECPHTVGLGQGYIIGSTELGLISIEAASDIKLESSCNFDLHTTSMAQKSFTQVEWRKKSDTTDTTNAASTTFEAQTKTVNLRGTCILAPELYDTLKKVKKTVLCYDLTCNQTHCQPTVYLIAPVLTCMSIRSCMASVFTSRIQVIYEKTHCVTGQLIEGQCFNPAHTLTLSQPAHTYDTVTLPISCFFTPKKSEQLKVIKTFEGILTKTGCTENALQGYYVCFLGSHSEPLIVPSLEDIRSAEVVSRMLVHPRGEDHDAIQNSQSHLRIVGPITAKVPSTSSTDTLKGTAFAGVPMYSSLSTLVRNADPEFVFSPGIVPESNHSTCDKKTVPITWTGYLPISGEMEKVTGCTVFCTLAGPGASCEAYSENGIFNISSPTCLVNKVQRFRGSEQKINFICQRVDQDVVVYCNGQKKVILTKTLVIGQCIYTFTSLFSLMPDVAHSLAVELCVPGLHGWATVMLLSTFCFGWVLIPAVTLIILKCLRVLTFSCSHYTNESKFKFILEKVKIEYQKTMGSMVCDVCHHECETAKELESHRQSCINGQCPYCMTITEATESALQAHYSICKLTGRFQEALKKSLKKPEVKKGCYRTLGVFRYKSRCYVGLVWCLLLTCEIVIWAASA

SEQ ID NO: 5 - protein, 511 aa, synthetic construct
  REGION 1..511: note = recombinant polypeptide
MEGWYLVVLGVCYTLTLAMPKTIYETPLMESGWSDTAHGVGEIPMKTDLELDFSLPSSSSYSYRRKLTNPANKEESIPFHFQMEKQVIHAEIQPLGHWMDATFNIKTAFHCYGACQKYSYPWQTSKCFFEKDYQYETGWGCNPGDCPGVGTGCTACGVYLDKLKSVGKAYKIISLKYTRKVCIQLGTEQTCKHIDANDCLVTPSVKVCIVGTVSKLQPSDTLLFLGPLEQGGIILKQWCTTSCAFGDPGDIMSTPSGMRCPEHTGSFRKICGFATTPVCEYQGNTISGYKRMMATKDSFQSFNLTEPHITTNKLEWIDPDGNTRDHVNLVLNRDVSFQDLSDNPCKVDLHTQAIEGAWGSGVGFTLTCTVGLTECPSFMTSIKACDLAMCYGSTVTNLARGSNTVKVVGKGGHSGSSFKCCHDTDCSSEGLLASAPHLERVTGFNQIDSDKVYDDGAPPCTFKCWFTKSGEWLLGILNGNWIVVVVLVVILILSIIMFSVLCPRRGHKKTV

SEQ ID NO: 6 - DNA, 4687 bp, synthetic construct
  misc_feature 1..4687: note = artificial polynuceotide
  source 1..4687: mol_type = other DNA
gataatacgactcactatagggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgagctgaagatggaatgccctcacaccgtgggcctcggccagggatatatcattggcagcaccgagctgggcctgatcagcatcgaggctgcctccgacatcaagctggaatctagctgcaacttcgacctgcacaccacatccatggctcaaaaaagcttcacccaggtggaatggcggaagaagagcgacaccacagacacaactaacgctgccagcacaacctttgaagcccagaccaaaactgtgaatctgcggggcacctgtatcctggctcctgaactgtacgatacactgaagaaggtgaagaaaacagtgctttgttatgacctgacctgcaaccagacacactgccagcctacagtgtacctgatcgcccctgtgctcacatgcatgagcatcagaagttgcatggccagcgtgttcacaagcagaatccaggtgatctacgagaagacacactgcgtgaccggacagctcatcgagggccaatgtttcaaccccgcccacaccctgaccctgagccagcctgcccacacctacgacaccgtgacactgcctataagctgcttcttcaccccaaagaagagcgaacagctgaaggtgattaagacattcgagggcatcctgactaagaccggctgcacagaaaacgccctgcagggctactacgtgtgcttcctgggcagccactctgagcccctgatcgtgccctctctggaagacatcagatccgccgaagtggtatctagaatgcttgtgcacccccgtggcgaggatcacgatgccatccagaactcacagagccacctgcggatcgtgggccctatcaccgccaaggtgccttctaccagcagcaccgacaccctgaagggcacagcatttgccggcgtgccaatgtacagcagtctgagcacactggtcagaaacgcggaccctgaattcgtgttcagccccggcatcgtgcctgagagcaaccacagcacctgtgacaagaagaccgtgccgatcacctggaccggctacctgcctatctccggcgaaatggaaaaagttacaggatgcacggtgttctgcaccctggccggccccggagccagctgcgaggcctactccgagaacggaatcttcaatatcagcagccctacctgcctggtgaacaaggtgcagcggttccggggcagcgagcagaagatcaacttcatctgccagagagtggaccaggacgtggtggtctactgcaatggacagaaaaaggtgatcctgaccaagacactggtgatcggccagtgtatctacacatttaccagcctgttctccctgatgcctgatgtggcccacagcctggctgttgagctgtgtgtgcctggcctgcacggctgggccacggtgatgctgttaagcaccttctgcttcggctgggtcctgatccccgctgtgacactgattatccttaagtgcctgagagtgctgacattttcttgtagccactacaccaacgagtccaagttcaagttcatcctggagaaggtgaagatcgagtaccagaagaccatgggcagcatggtgtgcgacgtgtgtcaccacgagtgcgagactgccaaggaactcgaatcccacagacagagctgcatcaacggccagtgtccttactgcatgaccatcaccgaggctacagagtctgccctgcaggcccattacagcatctgcaagctgaccggtagattccaggaggccctgaagaaatctctgaaaaagcctgaggtgaaaaagggctgttaccgcaccctgggcgtgttcagatacaagagcagatgttacgtgggcttggtgtggtgcctgctgctgacttgtgagatcgtgatctgggccgccagcgccgaaacacccctgatggaaagcgggtggagtgacaccgcccacggagtgggagagatccccatgaagaccgatctggagctggacttcagcctgcctagctcctcaagctacagctacagacggaagctgaccaaccccgctaacaaagaggaaagcatcccgttccacttccagatggaaaagcaagtgatccacgctgagatccagcctctgggacactggatggacgctaccttcaatatcaagaccgcctttcactgctacggcgcctgccagaaatattcttacccctggcagacctccaagtgttttttcgagaaagactaccagtacgagacaggatggggttgcaaccctggcgactgccctggagtgggcaccggatgcaccgcctgcggtgtgtacctagataagctgaagtctgttgggaaggcctacaagatcatttctctgaagtacacaagaaaggtgtgcatccagctgggcaccgagcaaacctgcaagcacatcgatgctaacgactgtctcgtcacccccagtgtgaaggtgtgcatcgtgggcacagtgagcaaacttcagcctagcgacaccctgctgttcctgggaccacttgagcagggcggaatcatcctgaagcagtggtgcaccacctcctgcgccttcggcgatcctggcgacatcatgagcaccccttctggcatgagatgcccagagcacacaggcagcttcagaaagatctgtggcttcgccactacccccgtgtgtgagtaccaaggcaacaccatctctggatataagcggatgatggccaccaaggacagcttccaaagcttcaacctgactgaacctcatataacaacaaacaagctggagtggatcgacccggatggcaacaccagagaccacgtgaacctggtgctgaacagagatgtctccttccaggatctgagcgataacccctgtaaagtggacctccatacccaggccatcgagggcgcttggggcagcggcgttggctttaccctgacatgtaccgtgggactgaccgaatgccctagctttatgacatctatcaaagcctgtgatctggccatgtgctacggcagcaccgtgactaacctggctagaggctctaataccgtcaaggtggtgggcaaaggcggccactccggctctagctttaagtgctgccacgacaccgactgcagcagcgagggcctgctggccagcgccccacatctggagcgggtgaccggcttcaaccagatcgacagcgacaaggtatacgacgacggtgcccctccttgcacatttaagtgctggttcaccaagtccggcgagtggctgctgggcatccttaacggcaattggatcgtggtggtggtgctggtggtgatcctgatcctgtcaatcattatgttcagcgtgctgtgccctcggagaggccacaagaaaaccgtctgaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctaggatatcaaaaggatccaa

SEQ ID NO: 7 - DNA, 5503 bp, synthetic construct
  misc_feature 1..5503: note = artificial polynucleotide
  source 1..5503: mol_type = other DNA
gataatacgactcactatagggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgagctgaagatggaatgccctcacaccgtgggcctcggccagggatatatcattggcagcaccgagctgggcctgatcagcatcgaggctgcctccgacatcaagctggaatctagctgcaacttcgacctgcacaccacatccatggctcaaaaaagcttcacccaggtggaatggcggaagaagagcgacaccacagacacaactaacgctgccagcacaacctttgaagcccagaccaaaactgtgaatctgcggggcacctgtatcctggctcctgaactgtacgatacactgaagaaggtgaagaaaacagtgctttgttatgacctgacctgcaaccagacacactgccagcctacagtgtacctgatcgcccctgtgctcacatgcatgagcatcagaagttgcatggccagcgtgttcacaagcagaatccaggtgatctacgagaagacacactgcgtgaccggacagctcatcgagggccaatgtttcaaccccgcccacaccctgaccctgagccagcctgcccacacctacgacaccgtgacactgcctataagctgcttcttcaccccaaagaagagcgaacagctgaaggtgattaagacattcgagggcatcctgactaagaccggctgcacagaaaacgccctgcagggctactacgtgtgcttcctgggcagccactctgagcccctgatcgtgccctctctggaagacatcagatccgccgaagtggtatctagaatgcttgtgcacccccgtggcgaggatcacgatgccatccagaactcacagagccacctgcggatcgtgggccctatcaccgccaaggtgccttctaccagcagcaccgacaccctgaagggcacagcatttgccggcgtgccaatgtacagcagtctgagcacactggtcagaaacgcggaccctgaattcgtgttcagccccggcatcgtgcctgagagcaaccacagcacctgtgacaagaagaccgtgccgatcacctggaccggctacctgcctatctccggcgaaatggaaaaagttacaggatgcacggtgttctgcaccctggccggccccggagccagctgcgaggcctactccgagaacggaatcttcaatatcagcagccctacctgcctggtgaacaaggtgcagcggttccggggcagcgagcagaagatcaacttcatctgccagagagtggaccaggacgtggtggtctactgcaatggacagaaaaaggtgatcctgaccaagacactggtgatcggccagtgtatctacacatttaccagcctgttctccctgatgcctgatgtggcccacagcctggctgttgagctgtgtgtgcctggcctgcacggctgggccacggtgatgctgttaagcaccttctgcttcggctgggtcctgatccccgctgtgacactgattatccttaagtgcctgagagtgctgacattttcttgtagccactacaccaacgagtccaagttcaagttcatcctggagaaggtgaagatcgagtaccagaagaccatgggcagcatggtgtgcgacgtgtgtcaccacgagtgcgagactgccaaggaactcgaatcccacagacagagctgcatcaacggccagtgtccttactgcatgaccatcaccgaggctacagagtctgccctgcaggcccattacagcatctgcaagctgaccggtagattccaggaggccctgaagaaatctctgaaaaagcctgaggtgaaaaagggctgttaccgcaccctgggcgtgttcagatacaagagcagatgttacgtgggcttggtgtggtgcctgctgctgacttgtgagatcgtgatctgggccgccagcgcctgattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgaaacacccctgatggaaagcgggtggagtgacaccgcccacggagtgggagagatccccatgaagaccgatctggagctggacttcagcctgcctagctcctcaagctacagctacagacggaagctgaccaaccccgctaacaaagaggaaagcatcccgttccacttccagatggaaaagcaagtgatccacgctgagatccagcctctgggacactggatggacgctaccttcaatatcaagaccgcctttcactgctacggcgcctgccagaaatattcttacccctggcagacctccaagtgttttttcgagaaagactaccagtacgagacaggatggggttgcaaccctggcgactgccctggagtgggcaccggatgcaccgcctgcggtgtgtacctagataagctgaagtctgttgggaaggcctacaagatcatttctctgaagtacacaagaaaggtgtgcatccagctgggcaccgagcaaacctgcaagcacatcgatgctaacgactgtctcgtcacccccagtgtgaaggtgtgcatcgtgggcacagtgagcaaacttcagcctagcgacaccctgctgttcctgggaccacttgagcagggcggaatcatcctgaagcagtggtgcaccacctcctgcgccttcggcgatcctggcgacatcatgagcaccccttctggcatgagatgcccagagcacacaggcagcttcagaaagatctgtggcttcgccactacccccgtgtgtgagtaccaaggcaacaccatctctggatataagcggatgatggccaccaaggacagcttccaaagcttcaacctgactgaacctcatataacaacaaacaagctggagtggatcgacccggatggcaacaccagagaccacgtgaacctggtgctgaacagagatgtctccttccaggatctgagcgataacccctgtaaagtggacctccatacccaggccatcgagggcgcttggggcagcggcgttggctttaccctgacatgtaccgtgggactgaccgaatgccctagctttatgacatctatcaaagcctgtgatctggccatgtgctacggcagcaccgtgactaacctggctagaggctctaataccgtcaaggtggtgggcaaaggcggccactccggctctagctttaagtgctgccacgacaccgactgcagcagcgagggcctgctggccagcgccccacatctggagcgggtgaccggcttcaaccagatcgacagcgacaaggtatacgacgacggtgcccctccttgcacatttaagtgctggttcaccaagtccggcgagtggctgctgggcatccttaacggcaattggatcgtggtggtggtgctggtggtgatcctgatcctgtcaatcattatgttcagcgtgctgtgccctcggagaggccacaagaaaaccgtctgaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctaggatatcaaaaggatccaa

SEQ ID NO: 8 - DNA, 3226 bp, synthetic construct
  misc_feature 1..3226: note = artificial polynucleotide
  source 1..3226: mol_type = other DNA
gataatacgactcactatagggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgagctgaagatggaatgccctcacaccgtgggcctcggccagggatatatcattggcagcaccgagctgggcctgatcagcatcgaggctgcctccgacatcaagctggaatctagctgcaacttcgacctgcacaccacatccatggctcaaaaaagcttcacccaggtggaatggcggaagaagagcgacaccacagacacaactaacgctgccagcacaacctttgaagcccagaccaaaactgtgaatctgcggggcacctgtatcctggctcctgaactgtacgatacactgaagaaggtgaagaaaacagtgctttgttatgacctgacctgcaaccagacacactgccagcctacagtgtacctgatcgcccctgtgctcacatgcatgagcatcagaagttgcatggccagcgtgttcacaagcagaatccaggtgatctacgagaagacacactgcgtgaccggacagctcatcgagggccaatgtttcaaccccgcccacaccctgaccctgagccagcctgcccacacctacgacaccgtgacactgcctataagctgcttcttcaccccaaagaagagcgaacagctgaaggtgattaagacattcgagggcatcctgactaagaccggctgcacagaaaacgccctgcagggctactacgtgtgcttcctgggcagccactctgagcccctgatcgtgccctctctggaagacatcagatccgccgaagtggtatctagaatgcttgtgcacccccgtggcgaggatcacgatgccatccagaactcacagagccacctgcggatcgtgggccctatcaccgccaaggtgccttctaccagcagcaccgacaccctgaagggcacagcatttgccggcgtgccaatgtacagcagtctgagcacactggtcagaaacgcggaccctgaattcgtgttcagccccggcatcgtgcctgagagcaaccacagcacctgtgacaagaagaccgtgccgatcacctggaccggctacctgcctatctccggcgaaatggaaaaagttacaggatgcacggtgttctgcaccctggccggccccggagccagctgcgaggcctactccgagaacggaatcttcaatatcagcagccctacctgcctggtgaacaaggtgcagcggttccggggcagcgagcagaagatcaacttcatctgccagagagtggaccaggacgtggtggtctactgcaatggacagaaaaaggtgatcctgaccaagacactggtgatcggccagtgtatctacacatttaccagcctgttctccctgatgcctgatgtggcccacagcctggctgttgagctgtgtgtgcctggcctgcacggctgggccacggtgatgctgttaagcaccttctgcttcggctgggtcctgatccccgctgtgacactgattatccttaagtgcctgagagtgctgacattttcttgtagccactacaccaacgagtccaagttcaagttcatcctggagaaggtgaagatcgagtaccagaagaccatgggcagcatggtgtgcgacgtgtgtcaccacgagtgcgagactgccaaggaactcgaatcccacagacagagctgcatcaacggccagtgtccttactgcatgaccatcaccgaggctacagagtctgccctgcaggcccattacagcatctgcaagctgaccggtagattccaggaggccctgaagaaatctctgaaaaagcctgaggtgaaaaagggctgttaccgcaccctgggcgtgttcagatacaagagcagatgttacgtgggcttggtgtggtgcctgctgctgacttgtgagatcgtgatctgggccgccagcgcctgaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctaggatatcaaaaggatccaa

SEQ ID NO: 9 - DNA, 2806 bp, synthetic construct
  misc_feature 1..2806: note = artificial polynucleotide
  source 1..2806: mol_type = other DNA
gataatacgactcactatagggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgaaacacccctgatggaaagcgggtggagtgacaccgcccacggagtgggagagatccccatgaagaccgatctggagctggacttcagcctgcctagctcctcaagctacagctacagacggaagctgaccaaccccgctaacaaagaggaaagcatcccgttccacttccagatggaaaagcaagtgatccacgctgagatccagcctctgggacactggatggacgctaccttcaatatcaagaccgcctttcactgctacggcgcctgccagaaatattcttacccctggcagacctccaagtgttttttcgagaaagactaccagtacgagacaggatggggttgcaaccctggcgactgccctggagtgggcaccggatgcaccgcctgcggtgtgtacctagataagctgaagtctgttgggaaggcctacaagatcatttctctgaagtacacaagaaaggtgtgcatccagctgggcaccgagcaaacctgcaagcacatcgatgctaacgactgtctcgtcacccccagtgtgaaggtgtgcatcgtgggcacagtgagcaaacttcagcctagcgacaccctgctgttcctgggaccacttgagcagggcggaatcatcctgaagcagtggtgcaccacctcctgcgccttcggcgatcctggcgacatcatgagcaccccttctggcatgagatgcccagagcacacaggcagcttcagaaagatctgtggcttcgccactacccccgtgtgtgagtaccaaggcaacaccatctctggatataagcggatgatggccaccaaggacagcttccaaagcttcaacctgactgaacctcatataacaacaaacaagctggagtggatcgacccggatggcaacaccagagaccacgtgaacctggtgctgaacagagatgtctccttccaggatctgagcgataacccctgtaaagtggacctccatacccaggccatcgagggcgcttggggcagcggcgttggctttaccctgacatgtaccgtgggactgaccgaatgccctagctttatgacatctatcaaagcctgtgatctggccatgtgctacggcagcaccgtgactaacctggctagaggctctaataccgtcaaggtggtgggcaaaggcggccactccggctctagctttaagtgctgccacgacaccgactgcagcagcgagggcctgctggccagcgccccacatctggagcgggtgaccggcttcaaccagatcgacagcgacaaggtatacgacgacggtgcccctccttgcacatttaagtgctggttcaccaagtccggcgagtggctgctgggcatccttaacggcaattggatcgtggtggtggtgctggtggtgatcctgatcctgtcaatcattatgttcagcgtgctgtgccctcggagaggccacaagaaaaccgtctgaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctaggatatcaaaaggatccaa

SEQ ID NO: 10 - protein, 5 aa, synthetic construct
  REGION 1..5: note = artificial peptide
WAASA

SEQ ID NO: 11 - protein, 22 aa, synthetic construct
  REGION 1..22: note = artificial peptide
GSGATNFSLLKQAGDVEENPGP

SEQ ID NO: 12 - DNA, 66 bp, synthetic construct
  misc_feature 1..66: note = artificial polynucleotide
  source 1..66: mol_type = other DNA
ggaagcggagctactaacttcagcctgctgaagcaggctggagacgtggaggagaaccctggacct

SEQ ID NO: 13 - RNA, 3867 nt, synthetic construct
  misc_feature 1..3867: note = artificial polynucleotide
  source 1..3867: mol_type = other RNA
ctagtagtagactccgcaagaagaagcaaaaaattaaagaagtgagtttaaaatggaaggctggtacctggtcgtgctgggcgtgtgttacaccctaacactggccatgcccaagaccatctacgagctgaagatggaatgccctcacaccgtgggcctcggccagggatatatcattggcagcaccgagctgggcctgatcagcatcgaggctgcctccgacatcaagctggaatctagctgcaacttcgacctgcacaccacatccatggctcaaaaaagcttcacccaggtggaatggcggaagaagagcgacaccacagacacaactaacgctgccagcacaacctttgaagcccagaccaaaactgtgaatctgcggggcacctgtatcctggctcctgaactgtacgatacactgaagaaggtgaagaaaacagtgctttgttatgacctgacctgcaaccagacacactgccagcctacagtgtacctgatcgcccctgtgctcacatgcatgagcatcagaagttgcatggccagcgtgttcacaagcagaatccaggtgatctacgagaagacacactgcgtgaccggacagctcatcgagggccaatgtttcaaccccgcccacaccctgaccctgagccagcctgcccacacctacgacaccgtgacactgcctataagctgcttcttcaccccaaagaagagcgaacagctgaaggtgattaagacattcgagggcatcctgactaagaccggctgcacagaaaacgccctgcagggctactacgtgtgcttcctgggcagccactctgagcccctgatcgtgccctctctggaagacatcagatccgccgaagtggtatctagaatgcttgtgcacccccgtggcgaggatcacgatgccatccagaactcacagagccacctgcggatcgtgggccctatcaccgccaaggtgccttctaccagcagcaccgacaccctgaagggcacagcatttgccggcgtgccaatgtacagcagtctgagcacactggtcagaaacgcggaccctgaattcgtgttcagccccggcatcgtgcctgagagcaaccacagcacctgtgacaagaagaccgtgccgatcacctggaccggctacctgcctatctccggcgaaatggaaaaagttacaggatgcacggtgttctgcaccctggccggccccggagccagctgcgaggcctactccgagaacggaatcttcaatatcagcagccctacctgcctggtgaacaaggtgcagcggttccggggcagcgagcagaagatcaacttcatctgccagagagtggaccaggacgtggtggtctactgcaatggacagaaaaaggtgatcctgaccaagacactggtgatcggccagtgtatctacacatttaccagcctgttctccctgatgcctgatgtggcccacagcctggctgttgagctgtgtgtgcctggcctgcacggctgggccacggtgatgctgttaagcaccttctgcttcggctgggtcctgatccccgctgtgacactgattatccttaagtgcctgagagtgctgacattttcttgtagccactacaccaacgagtccaagttcaagttcatcctggagaaggtgaagatcgagtaccagaagaccatgggcagcatggtgtgcgacgtgtgtcaccacgagtgcgagactgccaaggaactcgaatcccacagacagagctgcatcaacggccagtgtccttactgcatgaccatcaccgaggctacagagtctgccctgcaggcccattacagcatctgcaagctgaccggtagattccaggaggccctgaagaaatctctgaaaaagcctgaggtgaaaaagggctgttaccgcaccctgggcgtgttcagatacaagagcagatgttacgtgggcttggtgtggtgcctgctgctgacttgtgagatcgtgatctgggccgccagcgccgaaacacccctgatggaaagcgggtggagtgacaccgcccacggagtgggagagatccccatgaagaccgatctggagctggacttcagcctgcctagctcctcaagctacagctacagacggaagctgaccaaccccgctaacaaagaggaaagcatcccgttccacttccagatggaaaagcaagtgatccacgctgagatccagcctctgggacactggatggacgctaccttcaatatcaagaccgcctttcactgctacggcgcctgccagaaatattcttacccctggcagacctccaagtgttttttcgagaaagactaccagtacgagacaggatggggttgcaaccctggcgactgccctggagtgggcaccggatgcaccgcctgcggtgtgtacctagataagctgaagtctgttgggaaggcctacaagatcatttctctgaagtacacaagaaaggtgtgcatccagctgggcaccgagcaaacctgcaagcacatcgatgctaacgactgtctcgtcacccccagtgtgaaggtgtgcatcgtgggcacagtgagcaaacttcagcctagcgacaccctgctgttcctgggaccacttgagcagggcggaatcatcctgaagcagtggtgcaccacctcctgcgccttcggcgatcctggcgacatcatgagcaccccttctggcatgagatgcccagagcacacaggcagcttcagaaagatctgtggcttcgccactacccccgtgtgtgagtaccaaggcaacaccatctctggatataagcggatgatggccaccaaggacagcttccaaagcttcaacctgactgaacctcatataacaacaaacaagctggagtggatcgacccggatggcaacaccagagaccacgtgaacctggtgctgaacagagatgtctccttccaggatctgagcgataacccctgtaaagtggacctccatacccaggccatcgagggcgcttggggcagcggcgttggctttaccctgacatgtaccgtgggactgaccgaatgccctagctttatgacatctatcaaagcctgtgatctggccatgtgctacggcagcaccgtgactaacctggctagaggctctaataccgtcaaggtggtgggcaaaggcggccactccggctctagctttaagtgctgccacgacaccgactgcagcagcgagggcctgctggccagcgccccacatctggagcgggtgaccggcttcaaccagatcgacagcgacaaggtatacgacgacggtgcccctccttgcacatttaagtgctggttcaccaagtccggcgagtggctgctgggcatccttaacggcaattggatcgtggtggtggtgctggtggtgatcctgatcctgtcaatcattatgttcagcgtgctgtgccctcggagaggccacaagaaaaccgtctgacaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggtactgcatgcacgcaatgctagctgcccctttcccgtcctgggtaccccgagtctcccccgacctcgggtcccaggtatgctcccacctccacctgccccactcaccacctctgctagttccagacacctccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 14 - RNA, 4683 nt, synthetic construct
  misc_feature 1..4683: note = artificial polynucleotide
  source 1..4683: mol_type = other RNA
ctagtagtagactccgcaagaagaagcaaaaaattaaagaagtgagtttaaaatggaaggctggtacctggtcgtgctgggcgtgtgttacaccctaacactggccatgcccaagaccatctacgagctgaagatggaatgccctcacaccgtgggcctcggccagggatatatcattggcagcaccgagctgggcctgatcagcatcgaggctgcctccgacatcaagctggaatctagctgcaacttcgacctgcacaccacatccatggctcaaaaaagcttcacccaggtggaatggcggaagaagagcgacaccacagacacaactaacgctgccagcacaacctttgaagcccagaccaaaactgtgaatctgcggggcacctgtatcctggctcctgaactgtacgatacactgaagaaggtgaagaaaacagtgctttgttatgacctgacctgcaaccagacacactgccagcctacagtgtacctgatcgcccctgtgctcacatgcatgagcatcagaagttgcatggccagcgtgttcacaagcagaatccaggtgatctacgagaagacacactgcgtgaccggacagctcatcgagggccaatgtttcaaccccgcccacaccctgaccctgagccagcctgcccacacctacgacaccgtgacactgcctataagctgcttcttcaccccaaagaagagcgaacagctgaaggtgattaagacattcgagggcatcctgactaagaccggctgcacagaaaacgccctgcagggctactacgtgtgcttcctgggcagccactctgagcccctgatcgtgccctctctggaagacatcagatccgccgaagtggtatctagaatgcttgtgcacccccgtggcgaggatcacgatgccatccagaactcacagagccacctgcggatcgtgggccctatcaccgccaaggtgccttctaccagcagcaccgacaccctgaagggcacagcatttgccggcgtgccaatgtacagcagtctgagcacactggtcagaaacgcggaccctgaattcgtgttcagccccggcatcgtgcctgagagcaaccacagcacctgtgacaagaagaccgtgccgatcacctggaccggctacctgcctatctccggcgaaatggaaaaagttacaggatgcacggtgttctgcaccctggccggccccggagccagctgcgaggcctactccgagaacggaatcttcaatatcagcagccctacctgcctggtgaacaaggtgcagcggttccggggcagcgagcagaagatcaacttcatctgccagagagtggaccaggacgtggtggtctactgcaatggacagaaaaaggtgatcctgaccaagacactggtgatcggccagtgtatctacacatttaccagcctgttctccctgatgcctgatgtggcccacagcctggctgttgagctgtgtgtgcctggcctgcacggctgggccacggtgatgctgttaagcaccttctgcttcggctgggtcctgatccccgctgtgacactgattatccttaagtgcctgagagtgctgacattttcttgtagccactacaccaacgagtccaagttcaagttcatcctggagaaggtgaagatcgagtaccagaagaccatgggcagcatggtgtgcgacgtgtgtcaccacgagtgcgagactgccaaggaactcgaatcccacagacagagctgcatcaacggccagtgtccttactgcatgaccatcaccgaggctacagagtctgccctgcaggcccattacagcatctgcaagctgaccggtagattccaggaggccctgaagaaatctctgaaaaagcctgaggtgaaaaagggctgttaccgcaccctgggcgtgttcagatacaagagcagatgttacgtgggcttggtgtggtgcctgctgctgacttgtgagatcgtgatctgggccgccagcgcctgattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgaaacacccctgatggaaagcgggtggagtgacaccgcccacggagtgggagagatccccatgaagaccgatctggagctggacttcagcctgcctagctcctcaagctacagctacagacggaagctgaccaaccccgctaacaaagaggaaagcatcccgttccacttccagatggaaaagcaagtgatccacgctgagatccagcctctgggacactggatggacgctaccttcaatatcaagaccgcctttcactgctacggcgcctgccagaaatattcttacccctggcagacctccaagtgttttttcgagaaagactaccagtacgagacaggatggggttgcaaccctggcgactgccctggagtgggcaccggatgcaccgcctgcggtgtgtacctagataagctgaagtctgttgggaaggcctacaagatcatttctctgaagtacacaagaaaggtgtgcatccagctgggcaccgagcaaacctgcaagcacatcgatgctaacgactgtctcgtcacccccagtgtgaaggtgtgcatcgtgggcacagtgagcaaacttcagcctagcgacaccctgctgttcctgggaccacttgagcagggcggaatcatcctgaagcagtggtgcaccacctcctgcgccttcggcgatcctggcgacatcatgagcaccccttctggcatgagatgcccagagcacacaggcagcttcagaaagatctgtggcttcgccactacccccgtgtgtgagtaccaaggcaacaccatctctggatataagcggatgatggccaccaaggacagcttccaaagcttcaacctgactgaacctcatataacaacaaacaagctggagtggatcgacccggatggcaacaccagagaccacgtgaacctggtgctgaacagagatgtctccttccaggatctgagcgataacccctgtaaagtggacctccatacccaggccatcgagggcgcttggggcagcggcgttggctttaccctgacatgtaccgtgggactgaccgaatgccctagctttatgacatctatcaaagcctgtgatctggccatgtgctacggcagcaccgtgactaacctggctagaggctctaataccgtcaaggtggtgggcaaaggcggccactccggctctagctttaagtgctgccacgacaccgactgcagcagcgagggcctgctggccagcgccccacatctggagcgggtgaccggcttcaaccagatcgacagcgacaaggtatacgacgacggtgcccctccttgcacatttaagtgctggttcaccaagtccggcgagtggctgctgggcatccttaacggcaattggatcgtggtggtggtgctggtggtgatcctgatcctgtcaatcattatgttcagcgtgctgtgccctcggagaggccacaagaaaaccgtctgacaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggtactgcatgcacgcaatgctagctgcccctttcccgtcctgggtaccccgagtctcccccgacctcgggtcccaggtatgctcccacctccacctgccccactcaccacctctgctagttccagacacctccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 15 - RNA, 4649 nt, synthetic construct
  misc_feature 1..4649: note = artificial polynucleotide
  source 1..4649: mol_type = other RNA
ggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgagctgaagatggaatgccctcacaccgtgggcctcggccagggatatatcattggcagcaccgagctgggcctgatcagcatcgaggctgcctccgacatcaagctggaatctagctgcaacttcgacctgcacaccacatccatggctcaaaaaagcttcacccaggtggaatggcggaagaagagcgacaccacagacacaactaacgctgccagcacaacctttgaagcccagaccaaaactgtgaatctgcggggcacctgtatcctggctcctgaactgtacgatacactgaagaaggtgaagaaaacagtgctttgttatgacctgacctgcaaccagacacactgccagcctacagtgtacctgatcgcccctgtgctcacatgcatgagcatcagaagttgcatggccagcgtgttcacaagcagaatccaggtgatctacgagaagacacactgcgtgaccggacagctcatcgagggccaatgtttcaaccccgcccacaccctgaccctgagccagcctgcccacacctacgacaccgtgacactgcctataagctgcttcttcaccccaaagaagagcgaacagctgaaggtgattaagacattcgagggcatcctgactaagaccggctgcacagaaaacgccctgcagggctactacgtgtgcttcctgggcagccactctgagcccctgatcgtgccctctctggaagacatcagatccgccgaagtggtatctagaatgcttgtgcacccccgtggcgaggatcacgatgccatccagaactcacagagccacctgcggatcgtgggccctatcaccgccaaggtgccttctaccagcagcaccgacaccctgaagggcacagcatttgccggcgtgccaatgtacagcagtctgagcacactggtcagaaacgcggaccctgaattcgtgttcagccccggcatcgtgcctgagagcaaccacagcacctgtgacaagaagaccgtgccgatcacctggaccggctacctgcctatctccggcgaaatggaaaaagttacaggatgcacggtgttctgcaccctggccggccccggagccagctgcgaggcctactccgagaacggaatcttcaatatcagcagccctacctgcctggtgaacaaggtgcagcggttccggggcagcgagcagaagatcaacttcatctgccagagagtggaccaggacgtggtggtctactgcaatggacagaaaaaggtgatcctgaccaagacactggtgatcggccagtgtatctacacatttaccagcctgttctccctgatgcctgatgtggcccacagcctggctgttgagctgtgtgtgcctggcctgcacggctgggccacggtgatgctgttaagcaccttctgcttcggctgggtcctgatccccgctgtgacactgattatccttaagtgcctgagagtgctgacattttcttgtagccactacaccaacgagtccaagttcaagttcatcctggagaaggtgaagatcgagtaccagaagaccatgggcagcatggtgtgcgacgtgtgtcaccacgagtgcgagactgccaaggaactcgaatcccacagacagagctgcatcaacggccagtgtccttactgcatgaccatcaccgaggctacagagtctgccctgcaggcccattacagcatctgcaagctgaccggtagattccaggaggccctgaagaaatctctgaaaaagcctgaggtgaaaaagggctgttaccgcaccctgggcgtgttcagatacaagagcagatgttacgtgggcttggtgtggtgcctgctgctgacttgtgagatcgtgatctgggccgccagcgccgaaacacccctgatggaaagcgggtggagtgacaccgcccacggagtgggagagatccccatgaagaccgatctggagctggacttcagcctgcctagctcctcaagctacagctacagacggaagctgaccaaccccgctaacaaagaggaaagcatcccgttccacttccagatggaaaagcaagtgatccacgctgagatccagcctctgggacactggatggacgctaccttcaatatcaagaccgcctttcactgctacggcgcctgccagaaatattcttacccctggcagacctccaagtgttttttcgagaaagactaccagtacgagacaggatggggttgcaaccctggcgactgccctggagtgggcaccggatgcaccgcctgcggtgtgtacctagataagctgaagtctgttgggaaggcctacaagatcatttctctgaagtacacaagaaaggtgtgcatccagctgggcaccgagcaaacctgcaagcacatcgatgctaacgactgtctcgtcacccccagtgtgaaggtgtgcatcgtgggcacagtgagcaaacttcagcctagcgacaccctgctgttcctgggaccacttgagcagggcggaatcatcctgaagcagtggtgcaccacctcctgcgccttcggcgatcctggcgacatcatgagcaccccttctggcatgagatgcccagagcacacaggcagcttcagaaagatctgtggcttcgccactacccccgtgtgtgagtaccaaggcaacaccatctctggatataagcggatgatggccaccaaggacagcttccaaagcttcaacctgactgaacctcatataacaacaaacaagctggagtggatcgacccggatggcaacaccagagaccacgtgaacctggtgctgaacagagatgtctccttccaggatctgagcgataacccctgtaaagtggacctccatacccaggccatcgagggcgcttggggcagcggcgttggctttaccctgacatgtaccgtgggactgaccgaatgccctagctttatgacatctatcaaagcctgtgatctggccatgtgctacggcagcaccgtgactaacctggctagaggctctaataccgtcaaggtggtgggcaaaggcggccactccggctctagctttaagtgctgccacgacaccgactgcagcagcgagggcctgctggccagcgccccacatctggagcgggtgaccggcttcaaccagatcgacagcgacaaggtatacgacgacggtgcccctccttgcacatttaagtgctggttcaccaagtccggcgagtggctgctgggcatccttaacggcaattggatcgtggtggtggtgctggtggtgatcctgatcctgtcaatcattatgttcagcgtgctgtgccctcggagaggccacaagaaaaccgtctgaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctag

SEQ ID NO: 16 - RNA, 5466 nt, synthetic construct
  misc_feature 1..5466: note = artificial polynucleotide
  source 1..5466: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgagctgaagatggaatgccctcacaccgtgggcctcggccagggatatatcattggcagcaccgagctgggcctgatcagcatcgaggctgcctccgacatcaagctggaatctagctgcaacttcgacctgcacaccacatccatggctcaaaaaagcttcacccaggtggaatggcggaagaagagcgacaccacagacacaactaacgctgccagcacaacctttgaagcccagaccaaaactgtgaatctgcggggcacctgtatcctggctcctgaactgtacgatacactgaagaaggtgaagaaaacagtgctttgttatgacctgacctgcaaccagacacactgccagcctacagtgtacctgatcgcccctgtgctcacatgcatgagcatcagaagttgcatggccagcgtgttcacaagcagaatccaggtgatctacgagaagacacactgcgtgaccggacagctcatcgagggccaatgtttcaaccccgcccacaccctgaccctgagccagcctgcccacacctacgacaccgtgacactgcctataagctgcttcttcaccccaaagaagagcgaacagctgaaggtgattaagacattcgagggcatcctgactaagaccggctgcacagaaaacgccctgcagggctactacgtgtgcttcctgggcagccactctgagcccctgatcgtgccctctctggaagacatcagatccgccgaagtggtatctagaatgcttgtgcacccccgtggcgaggatcacgatgccatccagaactcacagagccacctgcggatcgtgggccctatcaccgccaaggtgccttctaccagcagcaccgacaccctgaagggcacagcatttgccggcgtgccaatgtacagcagtctgagcacactggtcagaaacgcggaccctgaattcgtgttcagccccggcatcgtgcctgagagcaaccacagcacctgtgacaagaagaccgtgccgatcacctggaccggctacctgcctatctccggcgaaatggaaaaagttacaggatgcacggtgttctgcaccctggccggccccggagccagctgcgaggcctactccgagaacggaatcttcaatatcagcagccctacctgcctggtgaacaaggtgcagcggttccggggcagcgagcagaagatcaacttcatctgccagagagtggaccaggacgtggtggtctactgcaatggacagaaaaaggtgatcctgaccaagacactggtgatcggccagtgtatctacacatttaccagcctgttctccctgatgcctgatgtggcccacagcctggctgttgagctgtgtgtgcctggcctgcacggctgggccacggtgatgctgttaagcaccttctgcttcggctgggtcctgatccccgctgtgacactgattatccttaagtgcctgagagtgctgacattttcttgtagccactacaccaacgagtccaagttcaagttcatcctggagaaggtgaagatcgagtaccagaagaccatgggcagcatggtgtgcgacgtgtgtcaccacgagtgcgagactgccaaggaactcgaatcccacagacagagctgcatcaacggccagtgtccttactgcatgaccatcaccgaggctacagagtctgccctgcaggcccattacagcatctgcaagctgaccggtagattccaggaggccctgaagaaatctctgaaaaagcctgaggtgaaaaagggctgttaccgcaccctgggcgtgttcagatacaagagcagatgttacgtgggcttggtgtggtgcctgctgctgacttgtgagatcgtgatctgggccgccagcgcctgattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgaaacacccctgatggaaagcgggtggagtgacaccgcccacggagtgggagagatccccatgaagaccgatctggagctggacttcagcctgcctagctcctcaagctacagctacagacggaagctgaccaaccccgctaacaaagaggaaagcatcccgttccacttccagatggaaaagcaagtgatccacgctgagatccagcctctgggacactggatggacgctaccttcaatatcaagaccgcctttcactgctacggcgcctgccagaaatattcttacccctggcagacctccaagtgttttttcgagaaagactaccagtacgagacaggatggggttgcaaccctggcgactgccctggagtgggcaccggatgcaccgcctgcggtgtgtacctagataagctgaagtctgttgggaaggcctacaagatcatttctctgaagtacacaagaaaggtgtgcatccagctgggcaccgagcaaacctgcaagcacatcgatgctaacgactgtctcgtcacccccagtgtgaaggtgtgcatcgtgggcacagtgagcaaacttcagcctagcgacaccctgctgttcctgggaccacttgagcagggcggaatcatcctgaagcagtggtgcaccacctcctgcgccttcggcgatcctggcgacatcatgagcaccccttctggcatgagatgcccagagcacacaggcagcttcagaaagatctgtggcttcgccactacccccgtgtgtgagtaccaaggcaacaccatctctggatataagcggatgatggccaccaaggacagcttccaaagcttcaacctgactgaacctcatataacaacaaacaagctggagtggatcgacccggatggcaacaccagagaccacgtgaacctggtgctgaacagagatgtctccttccaggatctgagcgataacccctgtaaagtggacctccatacccaggccatcgagggcgcttggggcagcggcgttggctttaccctgacatgtaccgtgggactgaccgaatgccctagctttatgacatctatcaaagcctgtgatctggccatgtgctacggcagcaccgtgactaacctggctagaggctctaataccgtcaaggtggtgggcaaaggcggccactccggctctagctttaagtgctgccacgacaccgactgcagcagcgagggcctgctggccagcgccccacatctggagcgggtgaccggcttcaaccagatcgacagcgacaaggtatacgacgacggtgcccctccttgcacatttaagtgctggttcaccaagtccggcgagtggctgctgggcatccttaacggcaattggatcgtggtggtggtgctggtggtgatcctgatcctgtcaatcattatgttcagcgtgctgtgccctcggagaggccacaagaaaaccgtctgaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctag

SEQ ID NO: 17 - RNA, 3207 nt, synthetic construct
  misc_feature 1..3207: note = artificial polynucleotide
  source 1..3207: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgagctgaagatggaatgccctcacaccgtgggcctcggccagggatatatcattggcagcaccgagctgggcctgatcagcatcgaggctgcctccgacatcaagctggaatctagctgcaacttcgacctgcacaccacatccatggctcaaaaaagcttcacccaggtggaatggcggaagaagagcgacaccacagacacaactaacgctgccagcacaacctttgaagcccagaccaaaactgtgaatctgcggggcacctgtatcctggctcctgaactgtacgatacactgaagaaggtgaagaaaacagtgctttgttatgacctgacctgcaaccagacacactgccagcctacagtgtacctgatcgcccctgtgctcacatgcatgagcatcagaagttgcatggccagcgtgttcacaagcagaatccaggtgatctacgagaagacacactgcgtgaccggacagctcatcgagggccaatgtttcaaccccgcccacaccctgaccctgagccagcctgcccacacctacgacaccgtgacactgcctataagctgcttcttcaccccaaagaagagcgaacagctgaaggtgattaagacattcgagggcatcctgactaagaccggctgcacagaaaacgccctgcagggctactacgtgtgcttcctgggcagccactctgagcccctgatcgtgccctctctggaagacatcagatccgccgaagtggtatctagaatgcttgtgcacccccgtggcgaggatcacgatgccatccagaactcacagagccacctgcggatcgtgggccctatcaccgccaaggtgccttctaccagcagcaccgacaccctgaagggcacagcatttgccggcgtgccaatgtacagcagtctgagcacactggtcagaaacgcggaccctgaattcgtgttcagccccggcatcgtgcctgagagcaaccacagcacctgtgacaagaagaccgtgccgatcacctggaccggctacctgcctatctccggcgaaatggaaaaagttacaggatgcacggtgttctgcaccctggccggccccggagccagctgcgaggcctactccgagaacggaatcttcaatatcagcagccctacctgcctggtgaacaaggtgcagcggttccggggcagcgagcagaagatcaacttcatctgccagagagtggaccaggacgtggtggtctactgcaatggacagaaaaaggtgatcctgaccaagacactggtgatcggccagtgtatctacacatttaccagcctgttctccctgatgcctgatgtggcccacagcctggctgttgagctgtgtgtgcctggcctgcacggctgggccacggtgatgctgttaagcaccttctgcttcggctgggtcctgatccccgctgtgacactgattatccttaagtgcctgagagtgctgacattttcttgtagccactacaccaacgagtccaagttcaagttcatcctggagaaggtgaagatcgagtaccagaagaccatgggcagcatggtgtgcgacgtgtgtcaccacgagtgcgagactgccaaggaactcgaatcccacagacagagctgcatcaacggccagtgtccttactgcatgaccatcaccgaggctacagagtctgccctgcaggcccattacagcatctgcaagctgaccggtagattccaggaggccctgaagaaatctctgaaaaagcctgaggtgaaaaagggctgttaccgcaccctgggcgtgttcagatacaagagcagatgttacgtgggcttggtgtggtgcctgctgctgacttgtgagatcgtgatctgggccgccagcgcctgaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctaggatatcaaaaggatccaa

SEQ ID NO: 18 - RNA, 2787 nt, synthetic construct
  misc_feature 1..2787: note = artificial polynucleotide
  source 1..2787: mol_type = other RNA
gggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggaaggctggtacctggtcgtgctgggcgtgtgttacaccctaacactggccatgcccaagaccatctacgaaacacccctgatggaaagcgggtggagtgacaccgcccacggagtgggagagatccccatgaagaccgatctggagctggacttcagcctgcctagctcctcaagctacagctacagacggaagctgaccaaccccgctaacaaagaggaaagcatcccgttccacttccagatggaaaagcaagtgatccacgctgagatccagcctctgggacactggatggacgctaccttcaatatcaagaccgcctttcactgctacggcgcctgccagaaatattcttacccctggcagacctccaagtgttttttcgagaaagactaccagtacgagacaggatggggttgcaaccctggcgactgccctggagtgggcaccggatgcaccgcctgcggtgtgtacctagataagctgaagtctgttgggaaggcctacaagatcatttctctgaagtacacaagaaaggtgtgcatccagctgggcaccgagcaaacctgcaagcacatcgatgctaacgactgtctcgtcacccccagtgtgaaggtgtgcatcgtgggcacagtgagcaaacttcagcctagcgacaccctgctgttcctgggaccacttgagcagggcggaatcatcctgaagcagtggtgcaccacctcctgcgccttcggcgatcctggcgacatcatgagcaccccttctggcatgagatgcccagagcacacaggcagcttcagaaagatctgtggcttcgccactacccccgtgtgtgagtaccaaggcaacaccatctctggatataagcggatgatggccaccaaggacagcttccaaagcttcaacctgactgaacctcatataacaacaaacaagctggagtggatcgacccggatggcaacaccagagaccacgtgaacctggtgctgaacagagatgtctccttccaggatctgagcgataacccctgtaaagtggacctccatacccaggccatcgagggcgcttggggcagcggcgttggctttaccctgacatgtaccgtgggactgaccgaatgccctagctttatgacatctatcaaagcctgtgatctggccatgtgctacggcagcaccgtgactaacctggctagaggctctaataccgtcaaggtggtgggcaaaggcggccactccggctctagctttaagtgctgccacgacaccgactgcagcagcgagggcctgctggccagcgccccacatctggagcgggtgaccggcttcaaccagatcgacagcgacaaggtatacgacgacggtgcccctccttgcacatttaagtgctggttcaccaagtccggcgagtggctgctgggcatccttaacggcaattggatcgtggtggtggtgctggtggtgatcctgatcctgtcaatcattatgttcagcgtgctgtgccctcggagaggccacaagaaaaccgtctgaaaaaaaacaaaaaacaaaacggctattatgcgttaccggcgagacgctacggacttaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctaggatatcaaaaggatccaa